(19) United States
(12) Patent Application Publication
(10) Pub. No.: US 2021/0181085 A1
(43) Pub. Date: Jun. 17, 2021

(54) RAPID MEASUREMENT OF PLATELETS

(71) Applicant: Essenlix Corporation, Monmouth Junction, NJ (US)

(72) Inventors: Stephen CHOU, Princeton, NJ (US); Wei DING, East Windsor, NJ (US)

(73) Assignee: Essenlix Corporation, Monmouth Junction, NJ (US)

(21) Appl. No.: 16/759,028

(22) PCT Filed: Oct. 26, 2018
(86) PCT No.: PCT/US18/57873

§ 371 Date: Apr. 24, 2020

Related U.S. Application Data
(60) Provisional application No. 62/577,456, filed on Oct. 26, 2017.

(51) Int. Cl. G01N 15/14; G01N 33/49

(52) U.S. Cl. CPC G01N 15/1456; G01N 2015/0084; G01N 33/49

(57) ABSTRACT

Among other things, the present invention is related to devices and methods of performing biological and chemical assays, in particular, of platelets.

### CROSS REFERENCING

This application is a National Stage entry (§ 371) application of International Application No. PCT/US18/57873, filed on Oct. 26, 2018, which claims the benefit of U.S. Provisional Patent Application No. 62/577,456, filed on Oct. 26, 2017, the contents of which are relied upon and incorporated herein by reference in their entirety.

The entire disclosure of any publication or patent document mentioned herein is entirely incorporated by reference.

### FIELD

Among other things, the present invention is related to devices and methods of performing biological and chemical assays, in particular, of platelets.

### BACKGROUND

In biological and chemical assays, it is often difficult and inaccurate in viewing platelets in a blood sample (whole blood or partial blood sample, undiluted or diluted), while in many cases, it is important to determine both quantity and quality of platelets in a blood sample. For instance, in blood bank, often one needs to know quickly the quality of stored platelets to determine if the platelets can be used for transfusion. The present invention allows to measure the platelet quantity and quality quickly.

The present invention provides devices and methods for improved viewing and/or counting of the platelets, and also for determining the quality of the platelets in undiluted or slightly diluted whole blood, or other types of blood sample.

### SUMMARY

In certain embodiments of the present disclosure, a device for assessing platelet quality in a blood sample can comprise a first plate; a second plate; spacers; and a viability dye. In certain embodiments, the plates are movable relative to each other into different configurations, including an open configuration and a closed configuration. In certain embodiments, each of the plates has, on its respective sample surface, a sample contact area for contacting a blood sample that includes platelets. In certain embodiments, one or both of the plates comprises the spacers, and the spacers are fixed to the respective sample contact area. In certain embodiments, each of the spacers has a pillar shape, a flat top, a predetermined substantially uniform height in the range of 0.5 μm to 6 μm, and a predetermined constant inter-spacer distance. In certain embodiments, the viability dye is coated on one or both of the plates, on their respective sample surfaces, and configured to stain the non-viable platelets of the blood sample and generate an optical signal indicative of a viability state of each of the stained platelets upon exposure to predetermined first wavelengths of light. In certain embodiments, in the open configuration, the two plates are partially or entirely separated apart, the spacing between the plates is not regulated by the spacers, and the blood sample is deposited on one or both of the plates. In certain embodiments, in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers.

In certain embodiments of the present disclosure, a kit for assessing platelet quality in a blood sample can comprise a first plate; a second plate; spacers; and a viability dye. In certain embodiments, the plates are movable relative to each other into different configurations, including an open configuration and a closed configuration. In certain embodiments, each of the plates has, on its respective sample surface, a sample contact area for contacting a blood sample that includes platelets. In certain embodiments, one or both of the plates comprises the spacers, and the spacers are fixed to the respective sample contact area. In certain embodiments, the spacers have a predetermined substantially uniform height in the range of 0.5 μm to 6 μm, and a predetermined constant inter-spacer distance. In certain embodiments, the viability dye is configured to stain the non-viable platelets of the blood sample and generate an optical signal indicative of a viability state of each of the stained platelets upon exposure to predetermined first wavelengths of light. In certain embodiments, in the open configuration, the two plates are partially or entirely separated apart, the spacing between the plates is not regulated by the spacers, and the blood sample is deposited on one or both of the plates. In certain embodiments, in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers.

In certain embodiments of the present disclosure, a system for assessing platelet quality in a blood sample can comprise the device of any embodiment of the present disclosure. In certain embodiments of the present disclosure, a system for assessing platelet quality in a blood sample can comprise an imager, comprising a camera and a light source for imaging the platelets in the layer of uniform thickness upon exposure to wavelengths of light that include at least the predetermined first wavelengths. In certain embodiments of the present disclosure, a system for assessing platelet quality in a blood sample can comprise a processor, comprising electronics, signal processors, hardware and software for receiving and processing the images and identifying and analyzing the platelets in the images.

In certain embodiments of the present disclosure, a method of assessing platelet quality in a blood sample can comprise obtaining a blood sample that includes platelets. In certain embodiments of the present disclosure, a method of assessing platelet quality in a blood sample can comprise obtaining a first plate and a second plate that are movable relative to each other into different configurations including an open configuration and a closed configuration. In certain embodiments, each of the plates has a sample contact area on its respective sample surface for contacting the blood sample. In certain embodiments, one or both of the plates comprises spacers that are fixed to its sample contact area. In certain embodiments, the spacers have a predetermined substantially uniform height in the range of 0.5 μm to 2.5 μm, and a predetermined constant inter-spacer distance. In certain embodiments of the present disclosure, a method of assessing platelet quality in a blood sample can comprise depositing the sample on one or both of the plates when the plates are in an open configuration, wherein in the open configuration the two plates are partially or entirely separated apart and the spacing between the plates is not regulated by the spacers. In certain embodiments of the present disclosure, a method of assessing platelet quality in a blood sample can comprise mixing the blood sample with a viability dye that is configured to stain the non-viable platelets and generate an optical signal indicative of a viability state of each of the stained platelets upon exposure to predetermined first wavelengths of light. In certain embodiments of the present disclosure, a method of assessing platelet quality in a blood sample can comprise bringing the two plates together and pressing the plates into a closed configuration, wherein in the closed configuration at least part of the blood sample is compressed by the two plates into a layer of highly uniform thickness and the uniform thickness of the layer is confined by the sample contact areas of the two plates and is regulated by the spacers and the plates. In certain embodiments of the present disclosure, a method of assessing platelet quality in a blood sample can comprise, while the plates are at the closed configuration, acquiring a bright-field image of the platelets and an image of the optical signals of the platelets rendered by the viability dye in the layer of uniform thickness. In certain embodiments of the present disclosure, a method of assessing platelet quality in a blood sample can comprise identifying the platelets in the acquired images and assessing the quality of the platelets.

The device, kit, system, or method of any embodiment of the present disclosure, wherein the viability dye selected from the group consisting of Propidium Iodide, 7-AAD, Trypan blue, Calcein Violet AM, Calcein AM, Fixable Viability Dyes, SYTO9 and other nucleic acid dyes, Resazurin and Formazan (MTT/XTT), other mitochondrial dyes, and any combination thereof. The device, kit, system, or method of any embodiment of the present disclosure, wherein the viability dye is coated on the sample contact area of one or both of the plates and configured to, upon contacting the blood sample, be dissolved and diffuse in the blood sample. The device, kit, system, or method of any embodiment of the present disclosure, wherein the viability dye is fluorescently labeled and the optical signal is a fluorescent signal. The device, kit, system, or method of any embodiment of the present disclosure, wherein the viability dye is a colorant and renders the stained platelets of different viability states different colors.

The device, kit, system, or method of any embodiment of the present disclosure, wherein the optical signal is:

fluorescence;

light absorption, reflection, transmission, diffraction, scattering, or diffusion;

surface Raman scattering; or

any combination of i-iii.

The device, kit, system, or method of any embodiment of the present disclosure, wherein the height of the spacers is around 2 μm. The device, kit, system, or method of any embodiment of the present disclosure, wherein the height of the spacers is selected such that in the closed configuration, a substantial fraction of RBCs in the layer of uniform thickness are lysed, and a substantial fraction of the platelets in the layer of uniform thickness are not lysed. The device, kit, system, or method of any embodiment of the present disclosure, wherein the height of the spacers is in the range of 0.5 μm to 1.2 μm. The device, kit, system, or method of any embodiment of the present disclosure, wherein on one or both the sample contact areas, the respective plate further comprises a layer of a lysing agent that facilitates the lysing of RBCs, WBCs, or other cells in the blood sample. The device, kit, system, or method of any embodiment of the present disclosure, wherein one or both the sample contact areas of the respective plate further comprises a layer of a reagent for bio/chemical assay of the platelets. The device, kit, system, or method of any embodiment of the present disclosure, wherein the lysing agent is selected from the group consisting of ammonium chloride, organic quaternary ammonium surfactants, cyanide salts, and any combination thereof. The device, kit, system, or method of any embodiment of the present disclosure, wherein the substantial fraction is at least 51%, 60%, 70%, 80%, 90%, 95% or 99% of a component in the relevant volume of the sample. The device, kit, system, or method of any embodiment of the present disclosure, wherein the thickness variation of the layer of highly uniform thickness over the lateral area of the relevant volume is equal to or less than 40%, 30%, 20%, 15%, 10%, 7%, 5%, 3%, or 1%, or in a range between any of the two values, wherein the thickness variation is relative to the average thickness of the lateral area. The device, kit, system, or method of any embodiment of the present disclosure, wherein the area of the highly uniform layer is equal to or larger than 0.1 mm², 0.5 mm², 1 mm², 3 mm², 5 mm², 10 mm², 20 mm², 50 mm², 70 mm², 100 mm², 200 mm², 500 mm², 800 mm², 1000 mm², 2000 mm², 5000 mm², 10000 mm², 20000 mm², 50000 mm², or 100000 mm²; or in a range between any of the two values. The device, kit, system, or method of any embodiment of the present disclosure, wherein the blood sample is diluted or undiluted whole blood. The device, kit, system, or method of any embodiment of the present disclosure, wherein the blood sample is a partial blood sample. The device, kit, system, or method of any embodiment of the present disclosure, wherein the spacer height is equal to or less than 2 μm, 1.9 μm, 1.8 μm, 1.7 μm, 1.6 μm, 1.5 μm, 1.4 μm, 1.3 μm, 1.2 μm, 1.1 μm, 1.0 μm, 0.9 μm, 0.8 μm, 0.7 μm, 0.6 μm, 0.5 μm, 0.4 μm, 0.3 μm, or 0.2 μm, or in a range between any of the two values. The device, kit, system, or method of any embodiment of the present disclosure, wherein in the closed configuration, a substantial fraction of white blood cells (WBCs) in the relevant volume of the sample are lysed, and the spacer height is equal to or less than 1.0 μm, 0.9 μm, 0.8 μm, 0.7 μm, 0.6 μm, 0.5 μm, 0.4 μm, 0.3 μm, or 0.2 μm, or in a range between any of the two values. The device, kit, system, or method of any embodiment of the present disclosure, wherein at least one of the plates is transparent. The system of any embodiment of the present disclosure, wherein the camera and the processor are part of a mobile communication device. The system of any embodiment of the present disclosure, wherein the light source is an internal light source of the mobile communication device. The system of any embodiment of the present disclosure, wherein the light source is a light source external to the mobile communication device. The system of any embodiment of the present disclosure, wherein the mobile communication device is a mobile phone. The system of any embodiment of the present disclosure, further comprising a housing configured to hold the sample and to be mounted to the mobile communication device. The system of any embodiment of the present disclosure, wherein the housing comprises optics for facilitating the imaging and/or signal processing of the sample by the mobile communication device and a mount configured to hold the optics on the mobile communication device. The system of any of any embodiment of the present disclosure, wherein the mobile communication device is configured to communicate test results to a medical professional, a medical facility or an insurance company. The system of any embodiment of the present disclosure, wherein the mobile communication device is further configured to communicate information on the subject with the medical professional, medical facility or insurance company. The system of any embodiment of the present disclosure, wherein the mobile communication device is configured to receive a prescription, diagnosis or a recommendation from a medical professional. The system of any embodiment of the present disclosure, wherein the mobile communication device communicates with the remote location via a Wi-Fi or cellular network. The system of any embodiment of the present disclosure, wherein the mobile communication device is a mobile phone.

The method of any embodiment of the present disclosure, wherein step (f) comprises the steps of:
imaging the platelets in the layer of uniform thickness under bright-field illumination; and
imaging fluorescent signals of the platelets in the layer of uniform thickness rendered by the viability dye upon exposure to the first wavelengths of light.

The method of any embodiment of the present disclosure, wherein step (g) is performed by:
identifying and obtaining a total number of the platelets in a first area of the acquired images;
classifying the platelets in the first area, based on the optical signals from individual platelets rendered by the viability dye; and
quantifying the quality of the platelets by calculating the percentage of the platelets in each class over the total number.

The method of any embodiment of the present disclosure, wherein the step (i) is performed by processing and analyzing the acquired images of the platelets under bright-field illumination to identify and obtain a total number of the platelets in the first area of the bright filed images. The method of any embodiment of the present disclosure, wherein the identifying step in step (g) comprises processing and analyzing the images using algorithms for edge detection and circle detection. The method of any embodiment of the present disclosure, wherein step (g) is performed by a mobile communication device that is configured to either receive or process the image of the platelets, or both. The method of any embodiment of the present disclosure, wherein the viability dye is supplied separately and added into the blood sample before the blood sample is deposited on the plates. The method of any embodiment of the present disclosure, wherein the viability dye is coated on the sample contact area of one or both of the plates, and configured to, upon contacting the sample, be dissolved and diffuse in the sample. The device, kit, system, or method of any embodiment of the present disclosure, wherein the spacers have: a shape of pillar with substantially uniform cross-section and a flat top surface; a ratio of the width to the height equal or larger than one; a filling factor of equal to 1% or larger; and a product of the filling factor and the Young's modulus of the spacer is 2 MPa or larger, wherein the filling factor is the ratio of the spacer contact area to the total plate area. The device, kit, system, or method of any embodiment of the present disclosure, wherein the spacer height is in the range of 0.5 μm to 62.5 μm. The device, kit, system, or method of any embodiment of the present disclosure, wherein a portion of the spacers have a periodic spacing. The device, kit, system, or method of any embodiment of the present disclosure, wherein an average value of the uniform thickness of the layer is substantially the same as the uniform height of the spacer with a variation of less than 10%. The device, kit, system, or method of any embodiment of the present disclosure, wherein in the closed configuration at least 90% of the RBCs are lysed and at least 90% of the platelets are not lysed. The device, kit, system, or method of any embodiment of the present disclosure, wherein in the closed configuration at least 99% of the RBCs are lysed and at least 99% of the platelets are not lysed. The device, kit, system, or method of any embodiment of the present disclosure, wherein the variation of the layer of uniform thickness is less than 30 nm. The device, kit, system, or method of any embodiment of the present disclosure, wherein the spacers are pillars with a cross-sectional shape selected from the group consisting of round, polygonal, circular, square, rectangular, oval, elliptical, and any combination of the same. The device, kit, system, or method of any embodiment of the present disclosure, wherein the spacers have: a shape of pillar with substantially uniform cross-section and a flat top surface; a ratio of the width to the height equal or larger than one; a predetermined constant inter-spacer distance that is in the range of 10 μm to 200 μm; a filling factor of equal to 1% or larger; and a product of the filling factor and the Young's modulus of the spacer is 2 MPa or larger.

wherein the filling factor is the ratio of the spacer contact area to a total plate area. The device, kit, system, or method of any embodiment of the present disclosure, wherein pressing the plates into the closed configuration is conducted either in parallel or sequentially, the parallel pressing applies an external force on an intended area at the same time, and the sequential pressing applies an external force on a part of an intended area and gradually moves to another area. The device, kit, system, or method of any embodiment of the present disclosure, wherein the blood sample is analyzed by: illuminating at least part of the blood sample in the layer of uniform thickness; obtaining one or more images of the cells using a CCD or CMOS sensor; identifying the platelets in the image using a computer; and counting a number of platelets in an area of the image. The device, kit, system, or method of any embodiment of the present disclosure, wherein the layer of uniform thickness has a thickness uniformity of up to +/−5%. A device for quantifying platelets in a blood sample, comprising: a first plate; a second plate; spacers; and a staining dye, wherein: the plates are movable relative to each other into different configurations, including an open configuration and a closed configuration, each of the plates has, on its respective sample surface, a sample contact area for contacting a blood sample that includes platelets, one or both of the plates comprises the spacers, and the spacers are fixed to the respective sample contact area, the spacers have a predetermined substantially uniform height in the range of 0.2 μm to 6 μm, and a predetermined constant inter-spacer distance, and the staining dye is coated on one or both of the plates, on their respective sample surfaces, and configured to stain the platelets of the blood sample and generate an optical signal indicative of the total amount of platelets in the blood sample upon exposure to predetermined first wavelengths of light, wherein in the open configuration, the two plates are partially or entirely separated apart, the spacing between the plates is not regulated by the spacers, and the blood sample is deposited on one or both of the plates, and wherein in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers. A kit for quantifying platelets in a blood sample, comprising: a first plate; a second plate; spacers; and a staining dye, wherein: the plates are movable relative to each other into different configurations, including an open configuration and a closed configuration, each of the plates has, on its respective sample surface, a sample contact area for contacting a blood sample that includes platelets, one or both of the plates comprises the spacers, and the spacers are fixed to the respective sample contact area, the spacers have a predetermined substantially uniform height in the range of 0.2 μm to 6 μm, and a predetermined constant inter-spacer distance, and the staining dye is configured to stain the platelets of the blood sample and generate an optical signal indicative of the total amount of platelets in the blood sample upon exposure to predetermined first wavelengths of light, wherein in the open configuration, the two plates are partially or entirely separated apart, the spacing between the plates is not regulated by the spacers, and the blood sample is deposited on one or both of the plates, and wherein in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers. A system for quantifying platelets in a blood sample, comprising: the device of any embodiment of the present disclosure; an imager, comprising a camera and a light source for imaging the platelets in the layer of uniform thickness upon exposure to wavelengths of light that include at least the predetermined first wavelengths; and a processor, comprising electronics, signal processors, hardware and software for receiving and processing the images and identifying and analyzing the platelets in the images. A method of quantifying platelets in a blood sample, comprising the steps of: obtaining a blood sample that includes platelets; obtaining a first plate and a second plate that are movable relative to each other into different configurations including an open configuration and a closed configuration, wherein: each of the plates has a sample contact area on its respective sample surface for contacting the blood sample, and one or both of the plates comprises spacers that are fixed to its sample contact area, wherein the spacers have a predetermined substantially uniform height in the range of 0.2 μm to 6 μm, and a predetermined constant inter-spacer distance, depositing the sample on one or both of the plates when the plates are in an open configuration, wherein in the open configuration the two plates are partially or entirely separated apart and the spacing between the plates is not regulated by the spacers; before or after (c), mixing the blood sample with a staining dye that is configured to stain the platelets and generate an optical signal indicative of the total amount of platelets in the blood sample upon exposure to predetermined first wavelengths of light; after (c) and (d), bringing the two plates together and pressing the plates into a closed configuration, wherein in the closed configuration at least part of the blood sample is compressed by the two plates into a layer of highly uniform thickness and the uniform thickness of the layer is confined by the sample contact areas of the two plates and is regulated by the spacers and the plates; while the plates are at the closed configuration, acquiring a bright-field image of the platelets and an image of the optical signals of the platelets rendered by the platelet staining dye in the layer of uniform thickness; and identifying the platelets in the acquired images and assessing the quality of the platelets. The device, kit, system, or method of any embodiment of the present disclosure, wherein the spacers have a predetermined substantially uniform height in the range of 2 μm to 5 μm. The device, kit, system, or method of any embodiment of the present disclosure, wherein the spacers have a predetermined substantially uniform height of 5 μm. The device, kit, system, or method of any embodiment of the present disclosure, wherein the staining dye is a dye selected from the group consisting of Acridine orange, YOYO-1, and methylene blue. The device, kit, system, or method of any embodiment of the present disclosure, wherein the staining dye is Acridine orange. The device, kit, system, or method any embodiment of the present disclosure, wherein the Acridine orange has a concentration of 0.4 mg/mL. The device, kit, system, or method of any embodiment of the present disclosure, wherein the staining dye is YOYO-1. The device, kit, system, or method of any embodiment of the present disclosure, wherein the YOYO-1 has a concentration of 5 μM-20 μM. The device, kit, system, or method of any embodiment of the present disclosure, wherein the YOYO-1 has a concentration of 10 μM. The device, kit, system, or method of any embodiment of the present disclosure, wherein the staining dye is methylene blue. The device, kit, system, or method of any embodiment of the present disclosure, wherein the methylene blue has a concentration of 0.01%-0.05%. The device, kit, system, or method of any embodiment of the present disclosure, wherein the staining dye is coated in a 15 mm×15 mm array of droplets having a 0.65 mm period, each droplet having a 11 nL volume. The device, kit, system, or method of any embodiment of the present disclosure, wherein each droplet contains Acridine orange and 3-[hexadecyl(dimethyl)azaniumyl]propane-1-sulfonate. The device, kit, system, or method of any embodiment of the present disclosure, wherein each droplet contains a Acridine orange concentration of 0.4 mg/mL and a 3-[hexadecyl(dimethyl) azaniumyl]propane-1-sulfonate concentration of 0.15 mg/mL. The device, kit, system, or method of any embodiment of the present disclosure, wherein each droplet contains YOYO-1 and 3-[hexadecyl(dimethyl)azaniumyl]propane-1-sulfonate. The device, kit, system, or method of any embodiment of the present disclosure, wherein each droplet contains a YOYO-1 concentration of 5 μM-20 μM and a 3-[hexadecyl(dimethyl) azaniumyl]propane-1-sulfonate concentration of 0.5 mg/mL-2.0 mg/mL. The device, kit, system, or method of any embodiment of the present disclosure, wherein each droplet contains methylene blue and 3-[hexadecyl(dimethyl)azaniumyl]propane-1-sulfonate. The device, kit, system, or method of any embodiment of the present disclosure, wherein each droplet contains a methylene blue concentration of 0.01%-0.05% and a 3-[hexadecyl(dimethyl) azaniumyl]propane-1-sulfonate concentration of 0.5 mg/mL-2.0 mg/mL. The device, kit, system, or method of any embodiment of the present disclosure, further comprising a lysing agent configured to lyse red blood cells in the blood sample. The device, kit, system, or method of any embodiment of the present disclosure, wherein the lysing agent is coated on one or both of the plates. The device, kit, system, or method of any embodiment of the present disclosure, wherein the lysing agent is selected from the group consisting of ammonium chloride, organic quaternary ammonium surfactants, cyanide salts, and detergent. The device, kit, system, or method of any embodiment of the present disclosure, wherein the lysing agent is selected from at least one of ammonium chloride, organic quaternary ammonium surfactants, cyanide salts, detergent, or any combination thereof. The device, kit, system, or method of any embodiment of the present disclosure, wherein the detergent comprises 3-[hexadecyl(dimethyl)azaniumyl]propane-1-sulfonate. The device kit, system, or method of any embodiment of the present disclosure, wherein the 3-[hexadecyl(dimethyl)azaniumyl]propane-1-sulfonate has a concentration of 0.5 mg/mL-2.0 mg/mL. The device, kit, system, or method of any embodiment of the present disclosure, wherein the 3-[hexadecyl(dimethyl)azaniumyl]propane-1-sulfonate has a concentration of 1 mg/m L. The device, kit, system, or method of any embodiment of the present disclosure, wherein the images are analyzed by machine learning.

### BRIEF DESCRIPTION OF THE DRAWINGS

The skilled artisan will understand that the drawings, described below, are for illustration purposes only. The drawings are not intended to limit the scope of the present teachings in any way. Some of the drawings are not in scale. In the figures that present experimental data points, the lines that connect the data points are for guiding a viewing of the data only and have no other means.

FIG. 1 shows an embodiment of a generic QMAX (Q: quantification; M: magnifying; A: adding reagents; X: acceleration; also known as compressed regulated open flow (CROF)) device.

FIG. 2 shows exemplary fluorescent images of white blood cells (WBC) and platelets (PLT) in the platelet assays according to some exemplary embodiments of the present invention.

FIG. 3 shows an image of a result of the platelet test using the device having a fluorescence label and no RBC lysing agent.

FIG. 4 shows an image of a result of the platelet test using the device having a fluorescence label and RBC lysing agent.

FIG. 5 shows an image of a result of the platelet test using the device having a colorimetric label and RBC lysing agent.

FIG. 6A shows an image of a result of the platelet test using the device having a fluorescence label and no RBC lysing agent after 1 minute.

FIG. 6B shows an image of a result of the platelet test using the device having a fluorescence label and no RBC lysing agent after 30 minutes.

FIG. 6C shows an image of a result of the platelet test using the device having a fluorescence label and no RBC lysing agent after 180 minutes.

FIG. 7 shows a graph depicting the decay of staining dye over time. Note, the decay of the staining dye is significant after 30 minutes.

FIG. 8 shows a graph depicting the decay of staining dye over time. Note, the staining dye is stable for 10 minutes.

FIG. 9 shows a table comparing the platelet counting method using the device having a fluorescence label and no RBC lysing, the platelet counting method using the device having a fluorescent label with RBC lysing, and the platelet counting method using a commercial hemocytometer.

### DETAILED DESCRIPTION OF EXEMPLARY EMBODIMENTS

The following detailed description illustrates some embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

According to one aspect, the present invention provides: (a) two plates to compress a blood sample into a thin layer that has a thickness, and (b) after (a), imaging process to view and/or counting the platelets. Spacers are used to control the final sample thickness and hence to assist a determination of the platelet concentration.

Another aspect of the present invention provides a viability dye to differentially label platelets of different viability states.

Another aspect of the present invention provides a nucleic acid-selective dye to label platelets as a means for rapid quantitation of the platelet counts.

Another aspect of the present invention provides both a viability dye to differentially label platelets of different viability states as well as a nucleic acid-selective dye to label platelets as a means for rapid quantitation of platelet count. The simultaneous staining provides accurate platelet quality information in human blood samples.

Another aspect of the present invention provides a fast staining method for viability test, where the viability dye is coated on the plate(s), and the compression of the sample by the two plates into a thin layer speeds up and eases the staining process.

Another aspect of the present invention provides uniformity of gap size between the two plates, hence leading to uniform lysing of specific cell types (e.g. red blood cells) over a significant area.

Another aspect of the present invention provides selective lysis of one type of cells (e.g. red blood cells and/or white blood cells) in a blood sample, while leaving platelets in the sample intact.

Another aspect of the present invention provides a lysing agent coated on the surface of one or both of the plates to facilitate the lysing of red blood cells and/or white blood cells in the sample, and/or the unlysing of the platelets.

Another aspect of the present invention provides imaging technique to view/count the platelets in the sample in bright-filed mode and/or fluorescent mode.

Another aspect of the present invention provides implement image processing and analysis algorithms for the measurement of the platelets.

Another aspect of the present invention provides implement image processing artificial intelligence and/or machine learning algorithms for the measurement of the platelets.

Another aspect of the present invention provides mobile communication device to facilitate the imaging and counting, and in some cases, remote health monitoring of the user of the devices. In many fields and applications, such as blood transfusion field, donated blood is often stored for prolonged periods of time prior to being used on recipients. Quality of donated blood required to be monitored for its quality before transfusion to a target recipient. Blood quality ensures the good quality of the blood components in the blood sample, as well as the good quantity of red blood cells (RBC), white blood cells (WBC), and platelets. For purposes of this application platelet quality can be assessed by the number of platelets present in a blood sample, and/or the viability of the platelets present in the blood sample.

In humans, the platelet concentration range is between 1.5-4.5×10⁵ cells/μL. The low platelet concentration is 1.5×10⁵ cells/μL, the normal platelet concentration is 3.0×10⁵ cells/μL, and the high platelet concentration is above 4.5×10⁵ cells/μL.

The embodiments in these applications herein incorporated can be regarded in combination with one another or as a single invention, rather than as discrete and independent filings. Moreover, the exemplary embodiments disclosed herein are applicable to embodiments including but not limited to: bio/chemical assays, QMAX cards and systems, QMAX with hinges, notches, recessed edges and sliders, assays and devices with uniform sample thickness, smartphone detection systems, cloud computing designs, various detection methods, labels, capture agents and detection agents, analytes, diseases, applications, and samples; the various embodiments are disclosed, described, and/or referred to in the aforementioned applications, all of which are hereby incorporated in reference by their entireties.

The current invention relates to identifying, tracking, and/or monitoring of any device that can be imaged for certain analysis (e.g. bio/chemical assays). The QMAX card is disclosed

### 1. VISUALIZING AND COUNTING PLATELETS

### 1.1. General Device and Method for Platelet Analysis

It is one aspect of the present invention to provide a device for assessing platelet quality in a blood sample. In some embodiments, the device is a QMAX device.

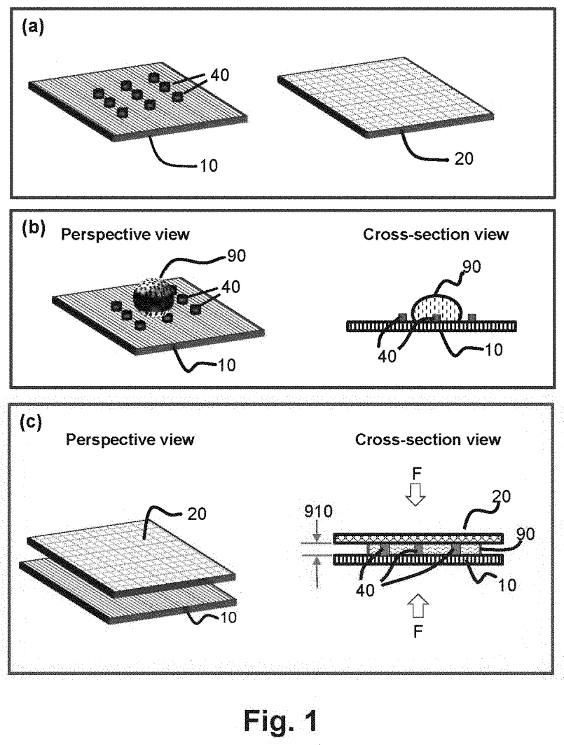

FIG. 1 shows an embodiment of a generic QMAX (Q: quantification; M: magnifying; A: adding reagents; X: acceleration; also known as compressed regulated open flow (CROF)) device. The generic QMAX device comprises a first plate 10 and a second plate 2. In particular, panel (A) shows the perspective view of a first plate 10 and a second plate 20 wherein the first plate has spacers. It should be noted, however, that the spacers can also be fixed on the second plate 20 (not shown) or on both first plate 10 and second plate 20 (not shown). Panel (B) shows the perspective view and a sectional view of depositing a sample 90 on the first plate 10 at an open configuration. It should be noted, however, that the sample 90 also can also be deposited on the second plate 20 (not shown), or on both the first plate 10 and the second plate 20 (not shown). Panel (C) illustrates (i) using the first plate 10 and second plate 20 to spread the sample 90 (the sample flow between the inner surfaces of the plates) and reduce the sample thickness, and (ii) using the spacers and the plate to regulate the sample thickness at the closed configuration of the QMAX device. The inner surfaces of each plate have one or a plurality of binding sites and or storage sites (not shown).

In some embodiments, the spacers 40 have a predetermined uniform height and a predetermined uniform inter-spacer distance. In the closed configuration, as shown in panel (C) of FIG. 1, the spacing between the plates and the thus the thickness of the sample 90 is regulated by the spacers 40. In some embodiments, the uniform thickness of the sample 90 is substantially similar to the uniform height of the spacers 40. It should be noted that although FIG. 1 shows the spacers 40 to be fixed on one of the plates, in some embodiments the spacers are not fixed. For example, in certain embodiments the spacers are mixed with the sample so that when the sample is compressed into a thin layer, the spacers, which is rigid beads or particles that have a uniform size, regulate the thickness of the sample layer.

In some embodiments, it is preferred to confine the platelets in the blood sample into a single layer in the layer of uniform thickness. In some embodiments, the spacing between the two plates at the closed configuration is 0.2 μm or more, 0.3 μm or more, 0.4 μm or more, 0.5 μm or more, 0.6 μm or more, 0.7 μm or more, 0.8 μm or more, 0.9 μm or more, 1 μm or more, 1.1 μm or more, 1.2 μm or more, 1.3 μm or more, 1.4 μm or more, 1.5 μm or more, 1.6 μm or more, 1.7 μm or more, 1.8 μm or more, 1.9 μm or more, 2.0 μm or more, 2.1 μm or more, 2.2 μm or more, 2.3 μm or more, 2.4 μm or more, 2.5 μm or more, or in a range between any two of these values.

In some embodiments, the spacing between the two plates at the closed configuration is equal to or approximately the uniform height of the spacers with a small deviation. In certain embodiments, the height of the spacers is in the range of 0.2 μm-6 μm. In certain preferred embodiments, the height of the spacers is in the range of 2 μm-5 μm. In certain preferred embodiments, the height of the spacer is 5 μm. In yet some preferred embodiments, the height of the spacers is 0.2 μm or more, 0.3 μm or more, 0.4 μm or more, 0.5 μm or more, 0.6 μm or more, 0.7 μm or more, 0.8 μm or more, 0.9 μm or more, 1 μm or more, 1.1 μm or more, 1.2 μm or more, 1.3 μm or more, 1.4 μm or more, 1.5 μm or more, 1.6 μm or more, 1.7 μm or more, 1.8 μm or more, 1.9 μm or more, 2.0 μm or more, 2.1 μm or more, 2.2 μm or more, 2.3 μm or more, 2.4 μm or more, 2.5 μm or more, or in a range between any two of these values.

In certain embodiments and applications, a sample thickness of 6 μm or less is preferred, because for a normal platelet concentration range in undiluted human blood, such thickness makes the platelet forming a single layer without substantial overlap, improving platelet counting accuracy or making platelet counting simpler (in many cases),

In some embodiments, the gap size of device is 0.2 μm, 0.4 μm, 1 μm, 2 μm, 5 μm, 10 μm, 20 μm, 30 μm, 40 μm, 50 μm or in a range between any of these two values.

In some embodiments, the gap size of device is in the range of 0.2 μm to 30 μm.

In some embodiments, the preferred gap size of device is in the range of 0.2 μm to 6 μm.

In some embodiments, the preferred gap size of device is 2 μm to 5 μm.

In some embodiments, the preferred gap size of device is 5 μm.

In some embodiments, the area of the highly uniform layer is equal to or larger than 0.1 mm², 0.5 mm², 1 mm², 3 mm², 5 mm², 10 mm², 20 mm², 50 mm², 70 mm², 100 mm², 200 mm², 500 mm², 800 mm², 1,000 mm², 2,000 mm², 5,000 mm², 10,000 mm², 20,000 mm², 50,000 mm², or 100,000 mm²; or in a range between any of the two values.

In some embodiments, the platelets are visualized under bright-field illumination. In some embodiments, the platelets are stained by a dye. In some embodiments, the dye is fluorescently labeled, therefore the stained platelets are visualized under fluorescent illumination.

In some embodiments, on one or both the sample contact areas, the respective plate further comprises a layer of a reagent for bio/chemical assay of the platelets. In some embodiments, one or both of the plates comprise, on the respective sample contact area, a dye for staining the platelets. In some embodiments, the dye is fluorescently labeled.

Mature platelets do not contain a nucleus, and do not have genomic DNA. However, platelets inherit a diverse array of functional coding or non-coding RNAs and translational machinery from their parent cells, enabling activated platelets to synthesize proteins, which suggests the possibility of post transcriptional gene regulation in platelets their RNA transcripts—needed for functional maintenance. Platelet RNAs can be derived from megakaryocytes during platelet origination. To detect platelets, we use fluorescent-labeled reagents that selective bind to RNAs in platelets.

In some embodiments, fluorescent images are taken of the platelets that are stained by fluorescently-labeled reagent. The fluorescently-labeled reagent is pre-loaded into the blood sample before being analyzed by QMAX device and/or coated on one or both of the plates of the QMAX device. Similar to the colorant as discussed above, in some embodiments, the fluorescently-labeled reagent differentially stains the platelets, for instance, it only stains the platelets, rendering only platelets in the sample emitting fluorescence upon stimulation, or it stains more substances besides platelets, but rendering the platelets emitting fluorescence with different parameters (e.g. excitation or emission spectra, intensity) than the surrounding substances. In some embodiments, the fluorescently-labeled reagent stains the platelets and other surrounding substances with no obvious difference. In some embodiments, the colorant is selected from the group consisting of: Acid fuchsin, Alcian blue 8 GX, Alizarin red S, Aniline blue WS, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, C.I. Cresyl violet, Crystal violet, Darrow red, Eosin B, Eosin Y, Erythrosin, Ethyl eosin, Ethyl green, Fast green F C F, Fluorescein Isothiocyanate, Giemsa Stain, Hematoxylin, Hematoxylin & Eosin, Indigo carmine, Janus green B, Jenner stain 1899, Light green SF, Malachite green, Martius yellow, Methyl orange, Methyl violet 2B, Methylene blue, Methylene blue, Methylene violet, (Bernthsen), Neutral red, Nigrosin, Nile blue A, Nuclear fast red, Oil Red, Orange G, Orange II, Orcein, Pararosaniline, Phloxin B, Protargol S, Pyronine B, Pyronine, Resazurin, Rose Bengal, Safranine O, Sudan black B, Sudan III, Sudan IV, Tetrachrome stain (MacNeal), Thionine, Toluidine blue, Weigert, Wright stain, and any combination thereof.

In some embodiments, the fluorescently-labeled reagent comprises fluorescent molecules (fluorophores), including, but not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, redshifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′,5″-dibromopyrogallol sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino- -fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; ophthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of 5 sulforhodamine (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; any combination thereof; and the like.

In some embodiments, fluorescently-labeled nucleic acid dyes are used to stain the platelets, which are capable of differentiating platelets from mature RBCs by highlighting the nuclei that exist in the former type of cells but not the latter. In some embodiments, these fluorescently-labeled nucleic acid dyes include, but not limited to, Acridine homodimer, Acridine orange, 7-AAD (7-amino-actinomycin D), Actinomycin D, ACMA, DAPI, Dihydroethidium, Ethidium bromide, Ethidium homodimer-1 (EthD-1), Ethidium homodimer-2 (EthD-2), Ethidium monoazide, Hexidium iodide, Hoechst 33258 (bis-benzimide), Hoechst 33342, Hoechst 34580, Hydroxystilbamidine, LDS 751, Nuclear yellow, Propidium iodide (PI); Quant-iT PicoGreen, Quant-iT OliGreen, SYBR Gold, SYBR Green I, SYBR Safe DNA stain, SYTOX Blue, SYTOX Green, SYTOX Orange, SYTOX Red, POPO-1, BOBO-1, YOYO-1, TOTO-1, JOJO-1, OPO-3, LOLO-1, BOBO-3, YOYO-3, TOTO-3, PO-PRO-1, YO-PRO-1, TO-PRO-1, JO-PRO-1, PO-PRO-3, YO-PRO-3, TO-PRO-3, TO-PRO-5, SYTO 40, SYTO 41, SYTO 42, SYTO 45, SYTO 81, SYTO 80, SYTO 82, SYTO 83, SYTO 84, SYTO 85, SYTO 64, SYTO 61, SYTO 17, SYTO 59, SYTO 62, SYTO 60, SYTO 63, and any combination thereof.

In certain preferred embodiments, the fluorescently-labeled nucleic acid dye is acridine orange. Acridine orange is a nucleic acid-selective fluorescent cationic dye. When bound to DNA, it is very similar spectrally to fluorescein, with an excitation maximum at 502 nm and an emission maximum at 525 nm (green). When it associates with RNA, the excitation maximum shifts to 460 nm (blue) and the emission maximum shifts to 650 nm (red). This dye can go into platelets and interact with DNA/RNA by intercalation or electrostatic attraction respectively. After acridine orange staining, platelets appear as bright greenish yellow bodies with a compact and almost centrally located inclusion, visible das a pale orange granule. Platelet staining with acridine orange often do not reveal a distinct sharp margin.

Acridine orange is derived from dimethylaminobenzaldehyde and N,N-dimethyl-1,3-diaminobenzene. Acridine dyes are prepared via the condensation of 1,3-diaminobenzene with suitable benzaldehydes.

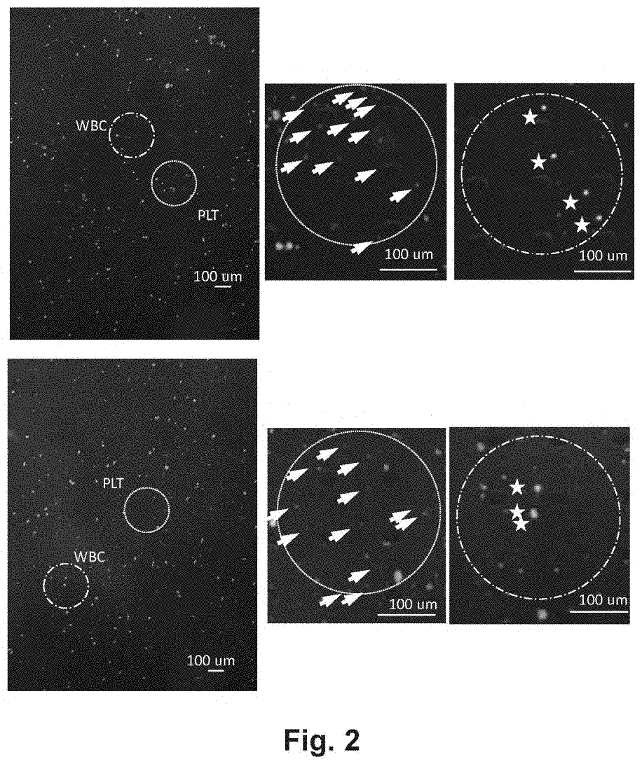

FIG. 2 shows exemplary fluorescent images of white blood cells (WBC) and platelets (PLT) in the platelet assays according to some exemplary embodiments of the present invention. The pictures in the middle and right columns are the zoomed-in images of the respective areas enclosed by the solid or dashed circles of the same sample, as shown in the respective pictures in the left column. In this exemplary assay, fresh blood sample was applied to the QMAX cards and compressed by the cards, while the WBCs and PLTs are stained by acridine orange that is coated on the inner surface of the QMAX cards. On the images, arrows indicate the PLTs, while stars indicate the WBCs, which are clearly larger in size and brighter in fluorescent intensity.

1.2 Selective Lysing of Red Blood Cells and/or White Blood Cells

In some embodiments, it is beneficial for platelet visualization and analysis to selectively lyse the abundant red blood cells and/or white blood cells in the blood sample. Detailed embodiments for selective lysing of RBCs and WBCs are disclosed in U.S. Provisional 62/539,672, which is cited herein as reference and incorporated in its entirety.

In some embodiments, the height of the spacers is selected such that in the closed configuration, a substantial fraction of RBCs in the layer of uniform thickness are lysed, and a substantial fraction of the platelets in the layer of uniform thickness are not lysed.

In some embodiments, the respective plate further comprises a layer of a reagent that facilitates: (a) the lysing of RBCs, WBCs, and/or other cells in the sample; and/or (b) the unlysing of platelets.

In some embodiments, the lysing agent is selected from either of ammonium chloride, organic quaternary ammonium surfactants, cyanide salts, zwittergent, or any combination thereof. In some preferred embodiments, the lysing agent is Zwittergent (or 3-[hexadecyl(dimethyl)azaniumyl]propane-1-sulfonate).

In certain embodiments, the lysing agents are coated on one plate (e.g., of the QMAX device). In certain embodiments, the lysing agents are coated on both plates of the QMAX device. In a preferred embodiment, the lysing agent (e.g., Zwittergent) is coated on the second plate of the QMAX device.

Coating can be achieved by printing the lysing agent onto the plate. An exemplary process used to coat lysing agent is droplet printing process. The lysing agents are configured to sufficiently lyse the other blood cell components such as red blood cells. The lysing agent is coated in a sufficient quantity to induce red blood cell lysis. Preferably, the lysing agent has a concentration of 0.5 mg/mL-2.0 mg/mL. More preferably, the lysing agent concentration is 1 mg/mL.

In some embodiments, the substantial fraction is at least 51%, 60%, 70%, 80%, 90%, 95% or 99% of a component in the relevant volume of the sample.

In some embodiments, the thickness variation of the layer of highly uniform thickness over the lateral area of the relevant volume is equal to or less than 40%, 30%, 20%, 15%, 10%, 7%, 5%, 3%, or 1%, or in a range between any of the two values, wherein the thickness variation is relative to the average thickness of the lateral area.

### 2. PLATELET VIABILITY TEST

### 2.1 Kit for Platelet Viability Test

It is another aspect of the present invention to provide a kit for assessing platelet quality in a blood sample.

In some embodiments, the kit comprises a device of the present invention, and a viability dye that is configured to stain the platelets and generate an optical signal indicative of viability state of individual platelets upon exposure to predetermined first wavelengths of light.

In some embodiments, the viability dye is separate from the device.

In some embodiments, the viability dye is coated on the sample contact area of one or both of the plates.

In some embodiments, both the viability dye (methylene blue) and the fluorescent dye (acridine orange) are coated on the sample contact area of one or both of the plates. Specifically, methylene blue (0.01-0.05%) and acridine orange (0.4 mg/mL) are coated on the sample contact area of one or both of the plates.

In some embodiments, the viability dye is configured to stain live platelets, but not dying or dead platelets. In some embodiments, the viability dye is configured to stain dying or dead platelets, but not live platelets. In some embodiments, the viability dye stains almost all platelets, but in different manners among platelets of different viability levels, thereby generating different optical signals of different platelets. For instance, the amount of dye incorporation may differ between live platelets and dead platelets. Or in some cases, the dye interacts with certain bio/chemical matters in platelets, which exist in different quantities or forms among platelets of different viability levels. Or in some cases, the degradation or other changes of cell membrane or intracellular membranous structures during cell death offers different dye incorporation patterns in dying and dead platelets than live platelets.

In some embodiments, the viability dye includes, but not limited to, Propidium Iodide, 7-AAD, Trypan blue, Calcein Violet AM, Calcein AM, Fixable Viability Dyes, SYTO9 and other nucleic acid dyes, Resazurin and Formazan (MTT/XTT) and other mitochondrial dyes, and any combination thereof.

In some embodiments, the viability dye is coated on the sample contact area of one or both of the plates, and configured to, upon contacting the sample, be dissolved and diffuse in the sample.

In some embodiments, the viability dye is fluorescently labeled, and the optical signal is fluorescent signal. In some embodiments, the viability dye is a colorant, and renders the stained platelets of different viability states different colors.

### 2.2 Method for Platelet Viability Test

It is another aspect of the present invention to provide a method for assessing platelet quality in a blood sample.

In some embodiments, the method comprises:

(a) obtaining a blood sample that includes platelets;

(b) obtaining a device provided by the present invention;

(c) depositing the sample on one or both of the plates when the plates are in an open configuration;

(d) before or after (c), mixing the sample with a viability dye that is configured to stain the platelets and generate an optical signal indicative of viability state of individual platelets upon exposure to predetermined first wavelengths of light;

(e) after (c) and (d), bringing the two plates together and pressing the plates into a closed configuration;

(f) while the plates are at the closed configuration, acquiring bright-field images of the platelets and images of the optical signals of the platelets rendered by the viability dye in the layer of uniform thickness; and

(g) identifying the platelets in the acquired images and analyzing the quality of the platelets.

In some embodiments, the viability dye is supplied separately and added into the blood sample before the sample is deposited on the plate(s). In some embodiments, the viability dye is coated on the sample contact area of one or both of the plates, and configured to, upon contacting the sample, be dissolved and diffuse in the sample. Therefore, the viability dye is added into the sample after the sample being deposited and touching the coated viability dye.

In some embodiments, the step (f) comprises: (i) imaging the platelets in the layer of uniform thickness under bright field illumination; and (ii) imaging fluorescent signals of platelets in the layer of uniform thickness rendered by the viability dye upon exposure to the first wavelengths of light.

In some embodiments, the step (g) comprises:

(i) identifying and obtaining a total number of the platelets in a first area of the acquired images;

(ii) classifying the platelets in the first area, based on the optical signals from individual platelets rendered by the viability dye; and

(iii) quantifying the quality of the platelets by calculating the percentage of the platelets in each class over the total number.

In some embodiments, the step (i) of identifying and obtaining comprises processing and analyzing images of the platelets acquired under bright field illumination, and obtaining a total number of the platelets in the first area of the bright filed images.

In some embodiments, the identifying in step (g) comprises processing and analyzing the images using algorithms for edge detection and circle detection.

### 3. SYSTEM FOR PLATELET VIABILITY TEST

It is another aspect of the present invention to provide a system for assessing platelet quality in a blood sample. In some embodiments, the system comprises: (a) a device of the present invention; (b) an imager, comprising a camera and a light source for imaging the platelets in the layer of uniform thickness upon exposure to wavelengths of light that include at least the first wavelengths; and (c) a processor, comprising electronics, signal processors, hardware and software for receiving and processing the images and identifying and analyzing the platelets in the images.

In some embodiments, the imager and the processor belong to a microscope system, which is configured to acquire and analyze images of the platelets under both bright-field and fluorescent modes. In some embodiments, the microscope system is equipped with appropriate light source, optical path (consisting of: e.g. excitation and emission filters, mirrors, condensers, imaging camera, and image data transportation, processing and analysis system).

In some embodiments, the imager and the camera and the processor are part of a mobile communication device. In some embodiments, the mobile communication device is a mobile phone.

In some embodiments, the light source is an internal light source of the mobile communication device. In some embodiments, the light source is a light source external to the mobile communication device.

In some embodiments, the system further comprises: (d) a housing configured to hold the sample and to be mounted to the mobile communication device. The term “housing” as used herein is interchangeable with the term “adaptor”. In some embodiments, the housing comprises optics for facilitating the imaging and/or signal processing of the sample by the mobile communication device, and a mount configured to hold the optics on the mobile communication device.

In some embodiments, the mobile communication device is configured to communicate test results to a medical professional, a medical facility or an insurance company. In some embodiments, the mobile communication device is further configured to communicate information on the subject with the medical professional, medical facility or insurance company. In some embodiments, the mobile communication device is configured to receive a prescription, diagnosis or a recommendation from a medical professional. In some embodiments, the mobile communication device communicates with the remote location via a Wi-Fi or cellular network.

In certain embodiments of the present disclosure, the sample deposition is a deposition directly from a subject to the plate without using any transferring devices. In certain embodiments, during the deposition, the amount of the sample deposited on the plate is unknown.

In certain embodiments, the method further comprises an analyzing that analyze the sample. In certain embodiments, the analyzing comprises calculating the volume of a relevant sample volume by measuring the lateral area of the relevant sample volume and calculating the volume from the lateral area and the predetermined spacer height. In certain embodiments, the pH value at location of a sample that is between the two plates in a closed configuration is determined by the volume of the location and by analyzing an image(s) taken from that location. In certain embodiments, the determination by analyzing an image uses artificial intelligence and machine learning Artificial Intelligence and/or Machine Learning to improve imaging

In certain embodiments of the present invention, the images taken during an assay operation and/or the samples measured by an assay are analyzed by artificial intelligence and machine learning. The samples include, but not limited to, medical samples, biology samples, environmental samples and chemistry samples.

In certain embodiments of the present invention, the sample is held by a QMAX device. The QMAX device together with imaging plus artificial intelligence and/or machine learning can overcome certain limitations in prior arts.

One important aspect of the present invention is to provide a machine learning framework to enhance the functionality, application scope and the accuracy in assaying using QMAX device, especially when a computer program is used.

In certain embodiments of the present invention, a device and a method for assaying sample and/or assay operation (e.g. tracking label identification) that utilizes QMAX together with imaging plus a machine learning and/or artificial intelligence comprises:

(1) using a QMAX device that has an auxiliary structure in the form of pillars to precisely control the distribution and volume of the sample in assaying, wherein the sample for assaying is loaded into the QMAX device and is kept between the two parallel plates on the QMAX device with an upper plate being transparent for imaging by an imager;

(2) the gap between the two parallel plates in the QMAX device is spaced narrowly—with the distance of the gap being proportional to the size of the analytes to be assayed—by which the analytes in the sample form a single layer between the said plates that can be imaged by an imager on the QMAX device;

(3) the sample volume corresponding to the AoI (area-of-interest) on the upper plate of the QMAX device can be precisely characterized by AoI and the gap—because of the uniformity of the gap between the plates in the QMAX device;

(4) the image on the sample for assaying sandwiched between the AoI x gap in the QMAX device is a pseudo-2D image, because it has the appearance of a 2D image, but it is an image of a 3D sample with its depth being known priori or characterized through other means;

(5) the captured pseudo-2D sample image taken over the AoI of the QMAX device can characterize the location of the analytes, color, shape, counts, and concentration of the analytes in the sample for assaying;

(6) based on abovementioned properties, the captured pseudo-2D image of QMAX device for assaying is amendable to a machine learning framework that applies to analyte detection, localization, identification, segmentation, counting, etc. for assaying in various applications; or

(7) any combination of thereof.

### 4. ARTIFICIAL INTELLIGENCE AND/OR MACHINE LEARNING FOR ANALYSIS

In certain embodiments of the present invention, a machine learning framework for QMAX based devices are implemented into a device that is capable of running an algorithms such as deep learning to discriminatively locate, identify, segment and count analytes (e.g. blood cells) based on the pseudo-2D image captured by the QMAX imager.

In certain embodiments of the present invention, the machine learning improves the images captured by the imager on the QMAX device and reduces the effects of noise and artifacts—including and not limited to air bobbles, dusts, shadows, and pillars.

In certain embodiments of the present invention, the training of machine learning uses the spacers of the QMAX card to reduce the data size of training set. Deep Learning. In certain embodiments, deep learning is used, wherein the analyte detection and localization workflow consists of two stages, training and prediction.

(i) Training Stage. At the training stage of the present invention, training data with annotation is fed into a convolutional neural network. Convolutional neural network is a specialized neural network for processing data that has a grid-like, feed forward and layered network topology. Examples of the data include time-series data, which can be thought of as a 1D grid taking samples at regular time intervals, and image data, which can be thought of as a 2D grid of pixels. Convolutional networks have been successful in practical applications. The name “convolutional neural network” indicates that the network employs a mathematical operation called convolution. Convolution is a specialized kind of linear operation. Convolutional networks are simply neural networks that use convolution in place of general matrix multiplication in at least one of their layers.

In training the machine learning model in some embodiments of the present invention, it receives one or multiple images of samples that contain the analytes taken by the imager over the sample holding QMAX device as training data. Training data are annotated for analytes to be assayed, wherein the annotations indicate whether or not analytes are in the training data and where they locate in the image. Annotation can be done in the form of tight bounding boxes which fully contains the analyte, or center locations of analytes. In the latter case, center locations are further converted into circles covering analytes or a Gaussian kernel in a point map.

When the size of training data is large, training machine learning model presents two challenges: annotation (usually done by human) is time consuming, and the training is computationally expensive. To overcome these challenges, one can partition the training data into patches of small size, then annotate and train on these patches, or a portion of these patches. The term “machine learning” refers to algorithms, systems and apparatus in the field of artificial intelligence that often use statistical techniques and artificial neural network trained from data without being explicitly programmed.

In some embodiments of the present invention, the annotated images are fed to the machine learning (ML) training module, and the model trainer in the machine learning module will train a ML model from the training data (annotated sample images). The input data will be fed to the model trainer in multiple iterations until certain stopping criterion is satisfied. The output of the ML training module is a ML model—a computational model that is built from a training process in the machine learning from the data that gives computer the capability to perform certain tasks (e.g. detect and classify the objects) on its own.

The trained machine learning model is applied during the predication (or inference) stage by the computer. Examples of machine learning models include ResNet, DenseNet, etc. which are also named as “deep learning models” because of the depth of the connected layers in their network structure. In some embodiments, the Caffe library with fully convolutional network (FCN) was used for model training and predication, and other convolutional neural network architecture and library can also be used, such as TensorFlow.

The training stage generates a model that will be used in the prediction stage. The model can be repeatedly used in the prediction stage for assaying the input. Thus, the computing unit only needs access to the generated model. It does not need access to the training data, nor requiring the training stage to be run again on the computing unit.

(ii) Prediction Stage. In the predication/inference stage, a detection component is applied to the input image, and an input image is fed into the predication (inference) module preloaded with a trained model generated from the training stage. The output of the prediction stage can be bounding boxes that contain the detected analytes with their center locations or a point map indicating the location of each analyte, or a heatmap that contains the information of the detected analytes.

When the output of the prediction stage is a list of bounding boxes, the number of analytes in the image of the sample for assaying is characterized by the number of detected bounding boxes. When the output of the prediction stage is a point map, the number of analytes in the image of the sample for assaying is characterized by the integration of the point map. When the output of the prediction is a heatmap, a localization component is used to identify the location and the number of detected analytes is characterized by the entries of the heatmap.

One embodiment of the localization algorithm is to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list.

In the detection component using heatmap, an input image, along with the model generated from the training stage, is fed into a convolutional neural network, and the output of the detection stage is a pixel-level prediction, in the form of a heatmap. The heatmap can have the same size as the input image, or it can be a scaled down version of the input image, and it is the input to the localization component. We disclose an algorithm to localize the analyte center. The main idea is to iteratively detect local peaks from the heatmap. After the peak is localized, we calculate the local area surrounding the peak but with smaller value. We remove this region from the heatmap and find the next peak from the remaining pixels. The process is repeated only all pixels are removed from the heatmap.

In certain embodiments, the present invention provides the localization algorithm to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list.

Algorithm GlobalSearch (heatmap)
Input:

heatmap

Output:

loci

loci ←{ }
sort(heatmap)
while (heatmap is not empty) {

s ← pop(heatmap)
D ← {disk center as s with radius R}
heatmap = heatmap \ D // remove D from the heatmap
add s to loc}

After sorting, heatmap is a one-dimensional ordered list, where the heatmap value is ordered from the highest to the lowest. Each heatmap value is associated with its corresponding pixel coordinates. The first item in the heatmap is the one with the highest value, which is the output of the pop(heatmap) function. One disk is created, where the center is the pixel coordinate of the one with highest heatmap value. Then all heatmap values whose pixel coordinates resides inside the disk is removed from the heatmap. The algorithm repeatedly pops up the highest value in the current heatmap, removes the disk around it, till the items are removed from the heatmap.

In the ordered list heatmap, each item has the knowledge of the proceeding item, and the following item. When removing an item from the ordered list, we make the following changes:
Assume the removing item is xr, its proceeding item is xp, and its following item is xf.
For the proceeding item xp, re-define its following item to the following item of the removing item. Thus, the following item of xp is now xf.
For the removing item xr, un-define its proceeding item and following item, which removes it from the ordered list.
For the following item xf, re-define its proceeding item to the proceeding item of the removed item. Thus, the proceeding item of xf is now xp.

After all items are removed from the ordered list, the localization algorithm is complete. The number of elements in the set loci will be the count of analytes, and location information is the pixel coordinate for each s in the set loci.

Another embodiment searches local peak, which is not necessary the one with the highest heatmap value. To detect each local peak, we start from a random starting point, and search for the local maximal value. After we find the peak, we calculate the local area surrounding the peak but with smaller value. We remove this region from the heatmap and find the next peak from the remaining pixels. The process is repeated only all pixels are removed from the heatmap.

Algorithm LocalSearch (s, heatmap)
Input:

s: starting location (x, y)
heatmap

Output:

s: location of local peak.

We only consider pixels of value > 0.
Algorithm Cover (s, heatmap)
Input:

s: location of local peak.
heatmap:

Output:

cover: a set of pixels covered by peak:

This is a breadth-first-search algorithm starting from s, with one altered condition of visiting points: a neighbor p of the current location q is only added to cover if heatmap[p]>0 and heatmap[p]<=heatmap[q]. Therefore, each pixel in cover has a non-descending path leading to the local peak s.

Algorithm Localization (heatmap)
Input:

heatmap

Output:

loci

loci ←{ }
pixels ←{all pixels from heatmap}
while pixels is not empty {

| s ←any pixel from pixels | |
|---|---|
| s ←LocalSearch(s, heatmap) // s is now local peak | |
| probe local region of radius R surrounding s for better local peak | |
| r ←Cover(s, heatmap) | |
| pixels ← pixels \ r | // remove all pixels in cover |

add s to loci

In certain embodiments, the image analysis comprising a Combination of Deep Learning and Computer Vision Approach, wherein I the detection and localization are realized by computer vision algorithms, and a classification is realized by deep learning algorithms, wherein the computer vision algorithms detect and locate possible candidates of analytes, and the deep learning algorithm classifies each possible candidate as a true analyte and false analyte. The location of all true analyte (along with the total count of true analytes) will be recorded as the output.

Detection. The computer vision algorithm detects possible candidate based on the characteristics of analytes, including but not limited to intensity, color, size, shape, distribution, etc. A pre-processing scheme can improve the detection. Pre-processing schemes include contrast enhancement, histogram adjustment, color enhancement, de-nosing, smoothing, de-focus, etc. After pre-processing, the input image is sent to a detector. The detector tells the existing of possible candidate of analyte and gives an estimate of its location. The detection can be based on the analyte structure (such as edge detection, line detection, circle detection, etc.), the connectivity (such as blob detection, connect components, contour detection, etc.), intensity, color, shape using schemes such as adaptive thresholding, etc.

Localization After detection, the computer vision algorithm locates each possible candidate of analytes by providing its boundary or a tight bounding box containing it. This can be achieved through object segmentation algorithms, such as adaptive thresholding, background subtraction, floodfill, mean shift, watershed, etc. Very often, the localization can be combined with detection to produce the detection results along with the location of each possible candidates of analytes.

Classification, the deep learning algorithms, such as convolutional neural networks, achieve start-of-the-art visual classification. We employ deep learning algorithms for classification on each possible candidate of analytes. Various convolutional neural network can be utilized for analyte classification, such as VGGNet, ResNet, MobileNet, DenseNet, etc.

Given each possible candidate of analyte, the deep learning algorithm computes through layers of neurons via convolution filters and non-linear filters to extract high-level features that differentiate analyte against non-analytes. A layer of fully convolutional network will combine high-level features into classification results, which tells whether it is a true analyte or not, or the probability of being an analyte.

### 5. ADVANTAGES OF THE PRESENT INVENTION

The present invention has many advantages over prior art methods. An advantage of the present invention is that the quantitation of platelets in blood sample can be performed using a platelet staining dye without any washing step. Another advantage is the fact that the amount of blood needed for the test is small (usually 1-5 μL). The test offers a performance time less than 5 min, preferably less than 1 min. The coupling of the iPhone and computer server enable the patient's platelet information be transmitted via the world-wide-web and establishes utility in the telemedicine field.

Unexpectedly, we observed that use of acridine orange to stain platelets require freshly isolated blood. Blood that has been isolated from blood circulation over a short period of time (e.g., less than 5-10 minutes) is optimal. On the other hand, blood that has been isolated from the blood circulation for a longer period of time (e.g., greater than 10 minutes) is not optimal. We observed that the acridine orange significantly loses (e.g., ˜50%) of its staining in the aged blood. The loss in platelet staining is not related to the inability of acridine orange to bind to DNA/RNA.

Without bound by a theory, it is believed that platelets in the aged blood may lose its DNA/RNA, for example, via enzymatic degradation. It is also possible that the minute amounts of DNA/RNA in platelets simply disintegrate and leave the cellular components.

It is an unexpected finding that the acridine orange staining is sensitive to the freshness of the blood drawn. The ability for acridine orange to stain platelets depends on the freshness of the blood (i.e., within 10 min after blood drawn). The staining is suboptimal towards aged blood. The present assay has application and utility to determine if the drawn blood is fresh.

We further observed that once the blood sample comes into contact with the acridine orange stain, the fluorescent stains will only decay slowly. It is of importance to perform acridine orange staining using fresh blood.

### 6. EXAMPLES OF PRESENT INVENTION

In one embodiment of the present disclosure, the present invention comprises a device for assessing platelet quality in a blood sample including a first plate, a second plate, and spacers, wherein the plates are movable relative to each other into different configurations, including an open configuration and a closed configuration. Each of the plates includes, on its respective sample surface, a sample contact area for contacting a blood sample that includes platelets. The spacers are disposed on either one or both of the plates and fixed to the respective sample contact area. The spacers have a predetermined substantially uniform height in the range of 0.5 μm to 2.5 μm and a predetermined constant inter-spacer distance. In the open configuration, the two plates are partially or entirely separated apart and the spacing between the plates is not regulated by the spacers. The sample is deposited on one or both of the plates when they are in the open configuration. In the closed configuration, which is configured after deposition of the sample in the open configuration at least part of the sample is compressed by the two plates into a layer of highly uniform thickness. The uniform thickness of the layer is confined by the sample surfaces of the plates and is regulated by the plates and the spacers.

In another embodiment of the present disclosure, the present invention comprises a kit for assessing platelet quality in a blood sample including a first plate, a second plate, spacers, and a viability dye, wherein the plates are movable relative to each other into different configurations, including an open configuration and a closed configuration. Each of the plates includes, on its respective sample surface, a sample contact area for contacting a blood sample that includes platelets. The spacers are disposed on either one or both of the plates and fixed to the respective sample contact area. The spacers have a predetermined substantially uniform height in the range of 0.5 μm to 2.5 μm and a predetermined constant inter-spacer distance. In the open configuration, the two plates are partially or entirely separated apart and the spacing between the plates is not regulated by the spacers. The sample is deposited on one or both of the plates when they are in the open configuration. In the closed configuration, which is configured after deposition of the sample in the open configuration at least part of the sample is compressed by the two plates into a layer of highly uniform thickness. The uniform thickness of the layer is confined by the sample surfaces of the plates and is regulated by the plates and the spacers. The viability dye is configured to stain the platelets and generate an optical signal indicative of viability state of individual platelets upon exposure to predetermined first wavelengths of light.

In yet another embodiment of the present disclosure, the present invention comprises a system for assessing platelet quality in a blood sample including a first plate, a second plate, spacers, an imager, and a processor, wherein the plates are movable relative to each other into different configurations, including an open configuration and a closed configuration. Each of the plates includes, on its respective sample surface, a sample contact area for contacting a blood sample that includes platelets. The spacers are disposed on either one or both of the plates and fixed to the respective sample contact area. The spacers have a predetermined substantially uniform height in the range of 0.5 μm to 2.5 μm and a predetermined constant inter-spacer distance. In the open configuration, the two plates are partially or entirely separated apart and the spacing between the plates is not regulated by the spacers.

The sample is deposited on one or both of the plates when they are in the open configuration. In the closed configuration, which is configured after deposition of the sample in the open configuration at least part of the sample is compressed by the two plates into a layer of highly uniform thickness. The uniform thickness of the layer is confined by the sample surfaces of the plates and is regulated by the plates and the spacers. The imager comprises a camera and a light source for imaging the platelets in the layer of uniform thickness upon exposure to wavelengths of light that include at least the first wavelengths. The processor comprises electronics, signal processors, hardware and software for receiving and processing the images and identifying and analyzing the platelets in the images.

In yet another embodiment of the present disclosure, the present invention comprises a method of assessing platelet quality in a blood sample including the steps of obtaining a blood sample that includes platelets, obtaining a first plate and a second plate, that are movable relative to each other into different configurations, including an open configuration and a closed configuration, wherein each of the plates has, on its respective sample surface, a sample contact area for contacting the sample, and wherein one or both of the plates comprises spacers that are fixed to a respective sample contact area. The spacers have a predetermined substantially uniform height in the range of 0.5 μm to 2.5 μm and a predetermined constant inter-spacer distance.

The method further comprises depositing the sample on one or both of the plates when the plates are in an open configuration, wherein in the open configuration the two plates are partially or entirely separated apart and the spacing between the plates is not regulated by the spacers, mixing the sample with a viability dye that is configured to stain the platelets and generate an optical signal indicative of viability state of individual platelets upon exposure to predetermined first wavelengths of light, bringing the two plates together and pressing the plates into a closed configuration, wherein in the closed configuration at least part of the sample is compressed by the two plates into a layer of highly uniform thickness, the uniform thickness of the layer is confined by the sample surfaces of the two plates and is regulated by the spacers and the plates, acquiring bright-field images of the platelets and images of the optical signals of the platelets rendered by the viability dye in the layer of uniform thickness; and identifying the platelets in the acquired images and analyzing the quality of the platelets.

In any embodiment of the present disclosure, the viability dye can comprise Propidium Iodide, 7-AAD, Trypan blue, Calcein Violet AM, Calcein AM, Fixable Viability Dyes, SYTO,9 or other nucleic acid dyes, or any combination thereof. In other embodiments of the present disclosure, the viability dye can comprise Resazurin and Formazan (MTT/XTT), or other mitochondrial dyes, or any combination thereof.

In any embodiment of the present disclosure, the viability dye can be fluorescently labeled. In any embodiment of the present disclosure, the viability dye can be a colorant, which renders the stained platelets of different viability states different colors.

In any embodiment of the present disclosure, the viability dye is disposed on one or both of the plates. In one embodiment, the viability dye is disposed on the sample contact area of one or both of the plates. In one embodiment, the viability dye is disposed on a portion of the sample contact area of one or both of the plates. In another embodiment, the viability dye is disposed on the entire sample contact area of one or both of the plates. In one embodiment, the viability dye is coated onto the sample contact area of one or both of the plates. In another embodiment, the viability dye is printed onto the sample contact area of one or both of the plates. In another embodiment, the viability dye is impregnated onto the sample contact area of one or both of the plates. Upon contacting a sample, the viability dye is configured to dissolve and diffuse in the sample.

In any embodiment of the present disclosure, the optical signal can be a fluorescent signal. In any embodiment of the present disclosure, the optical signal can comprise fluorescence, light absorption, reflection, transmission, diffraction, scattering, or diffusion, surface Raman scattering, or any combination thereof.

In any embodiment of the present disclosure, the height of the spacers can include a height within the range of 1 μm to 50 μm. In any embodiment of the present disclosure, the height of the spacers can include a height in the range of 2 μm to 30 μm. In any embodiment of the present disclosure, the height of the spacers can be 2 μm. In yet another embodiment of the present disclosure, the height of the spacers can include a range of 0.5 μm to 1.2 μm. In any embodiment of the present disclosure, the spacer height can be equal to or less than 2 μm, 1.9 μm, 1.8 μm, 1.7 μm, 1.6 μm, 1.5 μm, 1.4 μm, 1.3 μm, 1.2 μm, 1.1 μm, 1.0 μm, 0.9 μm, 0.8 μm, 0.7 μm, 0.6 μm, 0.5 μm, 0.4 μm, 0.3 μm, or 0.2 μm, or in a range between any of the two values.

In any embodiment of the present disclosure, the height of the spacers can be selected such that in the closed configuration, a substantial fraction of RBCs in the layer of uniform thickness are lysed, and a substantial fraction of the platelets in the layer of uniform thickness are not lysed.

In any embodiment of the present disclosure, the sample contact area of one or both of the plates can comprise a layer of a lysing agent that facilitates the lysing of RBCs, WBCs, and/or other cells in the sample and/or the unlysing of platelets. “Unlysing” as defined herein means that the platelets are not lysed. In any embodiment of the present disclosure, the sample contact areas of either one or both of the plates can comprises a layer of a reagent for bio/chemical assay of the platelets. In one embodiment, the lysing agent is coated onto the sample contact area of one or both of the plates. In another embodiment, the lysing agent is printed onto the sample contact area of one or both of the plates. In yet another embodiment, the lysing agent is disposed on a portion of the sample contact area. In yet another embodiment, the lysing agent is disposed on the entire sample contact area of one or both of the plates.

In any embodiment of the present disclosure, the lysing agent can be a lysing agent selected from the group consisting of ammonium chloride, organic quaternary ammonium surfactants, cyanide salts, and any combination thereof.

In any embodiment of the present disclosure, the substantial fraction of either the platelets or RBCs can be at least 51%, 60%, 70%, 80%, 90%, 95% or 99% of a component in the relevant volume of the sample.

In any embodiment of the present disclosure, the thickness variation of the layer of highly uniform thickness over the lateral area of the relevant volume can be equal to or less than 40%, 30%, 20%, 15%, 10%, 7%, 5%, 3%, or 1%, or in a range between any of the two values, wherein the thickness variation is relative to the average thickness of the lateral area.

In any embodiment of the present disclosure, the area of the highly uniform layer can be equal to or larger than 0.1 mm², 0.5 mm², 1 mm², 3 mm², 5 mm², 10 mm², 20 mm², 50 mm², 70 mm², 100 mm², 200 mm², 500 mm², 800 mm², 1000 mm², 2000 mm², 5000 mm², 10000 mm², 20000 mm², 50000 mm², or 100000 mm²; or in a range between any of the two values.

In any embodiment of the present disclosure, the blood sample can be diluted or undiluted whole blood. In any embodiment of the present disclosure, the blood sample can be a partial blood sample.

In any embodiment of the present disclosure, in the closed configuration, a substantial fraction of white blood cells (WBCs) in the relevant volume of the sample can be lysed, and the spacer height can be equal to or less than 1.0 μm, 0.9 μm, 0.8 μm, 0.7 μm, 0.6 μm, 0.5 μm, 0.4 μm, 0.3 μm, or 0.2 μm, or in a range between any of the two values.

In any embodiment of the present disclosure, one of the plates can be transparent. In any embodiment of the present disclosure, both plates can be transparent.

In any embodiment of the present disclosure, the camera and the processor can be a part of an electronic device, such as a tablet, computer, or smartphone. In another embodiment, the camera and processor can be a part of a mobile communication device. In any embodiment of the present disclosure, the mobile communication device can be a mobile phone, such as smart phone.

In any embodiment of the present disclosure, the light source can be an internal light source of the mobile communication device.

In any embodiment of the present disclosure, the light source can be a light source external to the mobile communication device.

In any embodiment of the present disclosure, the present invention can further comprise a housing configured to hold the sample and to be mounted to the mobile communication device. In any embodiment of the present disclosure, the housing can comprise a bracket for removably mounting the housing to the mobile communication device and/or electronic device. In any embodiment of the present disclosure, the housing can comprise optics for facilitating the imaging and/or signal processing of the sample by the mobile communication device, and a mount configured to hold the optics on the mobile communication device.

In any embodiment of the present disclosure, the mobile communication device can be configured to communicate test results to a medical professional, a medical facility or an insurance company.

In any embodiment of the present disclosure, the mobile communication device can be configured to communicate information on the subject with the medical professional, medical facility or insurance company.

In any embodiment of the present disclosure, the mobile communication device can be configured to receive a prescription, diagnosis or a recommendation from a medical professional.

In any embodiment of the present disclosure, the mobile communication device can communicate with the remote location via a Wi-Fi or cellular network.

In any embodiment of the present disclosure, step (f) can comprise the steps of imaging the platelets in the layer of uniform thickness under bright field illumination, and imaging fluorescent signals of platelets in the layer of uniform thickness rendered by the viability dye upon exposure to the first wavelengths of light.

In any embodiment of the present disclosure, step (g) can comprise the steps of identifying and obtaining a total number of the platelets in a first area of the acquired images, classifying the platelets in the first area, based on the optical signals from individual platelets rendered by the viability dye, and quantifying the quality of the platelets by calculating the percentage of the platelets in each class over the total number.

In any embodiment of the present disclosure, step (i) of step (g) can comprise processing and analyzing images of the platelets acquired under bright field illumination, and obtaining a total number of the platelets in the first area of the bright filed images.

In any embodiment of the present disclosure, the identifying step in step (g) can comprise processing and analyzing the images using algorithms for edge detection and circle detection.

In any embodiment of the present disclosure, the identifying and analyzing step of step (g) can be performed by a mobile communication device that is configured to receive and/or process the image of the platelets.

In any embodiment of the present disclosure, the spacers can comprise a shape of pillar with a substantially uniform cross-section and a flat top surface, wherein a ratio of the width to the height equal or larger than one, a filling factor that is equal to 1% or larger, wherein a product of the filling factor and the Young's modulus of the spacer is 2 MPa or larger. The filling factor is the ratio of the spacer contact area to the total plate area.

In any embodiment of the present disclosure, an average value of the uniform thickness of the layer can be substantially the same as the uniform height of the spacer with a variation of less than 10%.

In any embodiment of the present disclosure, in the closed configuration at least 90% of the RBCs can be lysed and at least 90% of the platelets can be not lysed.

In any embodiment of the present disclosure, in the closed configuration at least 99% of the RBCs can be lysed and at least 99% of the platelets can be not lysed. In any embodiment of the present disclosure, the variation of the layer of uniform thickness can be less than 30 nm.

In any embodiment of the present disclosure, the layer of uniform thickness sample can have a thickness uniformity of up to +/−5%.

In any embodiment of the present disclosure, the spacers can be pillars with a cross-sectional shape selected from the group consisting of round, polygonal, circular, square, rectangular, oval, elliptical, and any combination of the same.

In any embodiment of the present disclosure, the spacers can comprise a shape of pillar with substantially uniform cross-section and a flat top surface, wherein a ratio of the width to the height is equal to or larger than one, a predetermined constant inter-spacer distance that is in the range of 10 μm to 200 μm, a filling factor that is equal to 1% or larger, and wherein a product of the filling factor and the Young's modulus of the spacer is 2 MPa or larger. The filling factor is the ratio of the spacer contact area to a total plate area.

In any embodiment of the present disclosure, pressing the plates into the closed configuration can be conducted either in parallel or sequentially, wherein the parallel pressing applies an external force on an intended area at the same time, and the sequential pressing applies an external force on a part of an intended area and gradually moves to another area.

In any embodiment of the present disclosure, the blood sample can be analyzed by illuminating at least part of the blood sample in the layer of uniform thickness, obtaining one or more images of the cells using a CCD or CMOS sensor, identifying the platelets in the image using a computer, and counting a number of platelets in an area of the image.

### 7. ADDITIONAL EXAMPLES AND DEFINITIONS

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### (1) Definitions

The terms used in describing the devices/apparatus, systems, and methods herein disclosed are defined in the current application, or in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (2) Sample

The devices/apparatus, systems, and methods herein disclosed can be applied to manipulation and detection of various types of samples. The samples are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for samples such as but not limited to diagnostic samples, clinical samples, environmental samples and foodstuff samples. The types of sample include but are not limited to the samples listed, described and/or summarized in PCT Application (designating U.S.) Nos. PCT/US2016/046437 and PCT/US2016/051775, which were respectively filed on Aug. 10, 2016 and Sep. 14, 2016, and are hereby incorporated by reference by their entireties.

For example, in some embodiments, the devices, apparatus, systems, and methods herein disclosed are used for a sample that includes cells, tissues, bodily fluids and/or a mixture thereof. In some embodiments, the sample comprises a human body fluid. In some embodiments, the sample comprises at least one of cells, tissues, bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, and exhaled breath condensate.

In some embodiments, the devices, apparatus, systems, and methods herein disclosed are used for an environmental sample that is obtained from any suitable source, such as but not limited to: river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. In certain embodiments, the environmental sample is fresh from the source; in certain embodiments, the environmental sample is processed. For example, samples that are not in liquid form are converted to liquid form before the subject devices, apparatus, systems, and methods are applied.

In some embodiments, the devices, apparatus, systems, and methods herein disclosed are used for a foodstuff sample, which is suitable or has the potential to become suitable for animal consumption, e.g., human consumption. In some embodiments, a foodstuff sample includes raw ingredients, cooked or processed food, plant and animal sources of food, preprocessed food as well as partially or fully processed food, etc. In certain embodiments, samples that are not in liquid form are converted to liquid form before the subject devices, apparatus, systems, and methods are applied.

The subject devices, apparatus, systems, and methods can be used to analyze any volume of the sample. Examples of the volumes include, but are not limited to, about 10 mL or less, 5 mL or less, 3 mL or less, 1 microliter (μL, also “uL” herein) or less, 500 μL or less, 300 μL or less, 250 μL or less, 200 μL or less, 170 μL or less, 150 μL or less, 125 μL or less, 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values.

In some embodiments, the volume of the sample includes, but is not limited to, about 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values. In some embodiments, the volume of the sample includes, but is not limited to, about 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values.

In some embodiments, the amount of the sample is about a drop of liquid. In certain embodiments, the amount of sample is the amount collected from a pricked finger or fingerstick. In certain embodiments, the amount of sample is the amount collected from a microneedle, micropipette or a venous draw.

In certain embodiments, the sample holder is configured to hold a fluidic sample. In certain embodiments, the sample holder is configured to compress at least part of the fluidic sample into a thin layer. In certain embodiments, the sample holder comprises structures that are configured to heat and/or cool the sample. In certain embodiments, the heating source provides electromagnetic waves that can be absorbed by certain structures in the sample holder to change the temperature of the sample. In certain embodiments, the signal sensor is configured to detect and/or measure a signal from the sample. In certain embodiments, the signal sensor is configured to detect and/or measure an analyte in the sample. In certain embodiments, the heat sink is configured to absorb heat from the sample holder and/or the heating source. In certain embodiments, the heat sink comprises a chamber that at least partly enclose the sample holder.

### (3) Q-Card, Spacers and Uniform Sample Thickness

The devices/apparatus, systems, and methods herein disclosed can include or use Q-cards, spacers, and uniform sample thickness embodiments for sample detection, analysis, and quantification. In some embodiments, the Q-card comprises spacers, which help to render at least part of the sample into a layer of high uniformity. The structure, material, function, variation and dimension of the spacers, as well as the uniformity of the spacers and the sample layer, are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (4) Hinges, Opening Notches, Recessed Edge and Sliders

The devices/apparatus, systems, and methods herein disclosed can include or use Q-cards for sample detection, analysis, and quantification. In some embodiments, the Q-card comprises hinges, notches, recesses, and sliders, which help to facilitate the manipulation of the Q card and the measurement of the samples. The structure, material, function, variation and dimension of the hinges, notches, recesses, and sliders are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/431,639, which was filed on Dec. 9, 2016, U.S. Provisional Application No. 62/456,065, which was filed on Feb. 7, 2017, U.S. Provisional Application Nos. 62/456,287 and 62/456,504, which was filed on Feb. 8, 2017, and U.S. Provisional Application No. 62/539,660, which was filed on Aug. 1, 2017, all of which applications are incorporated herein in their entireties for all purposes.

In some embodiments, the QMAX device comprises opening mechanisms such as but not limited to notches on plate edges or strips attached to the plates, making is easier for a user to manipulate the positioning of the plates, such as but not limited to separating the plates of by hand.

In some embodiments, the QMAX device comprises trenches on one or both of the plates. In certain embodiments, the trenches limit the flow of the sample on the plate.

### (5) Q-Card and Adaptor

The devices/apparatus, systems, and methods herein disclosed can include or use Q-cards for sample detection, analysis, and quantification. In some embodiments, the Q-card is used together with an adaptor that is configured to accommodate the Q-card and connect to a mobile device so that the sample in the Q-card can be imaged, analyzed, and/or measured by the mobile device. The structure, material, function, variation, dimension and connection of the Q-card, the adaptor, and the mobile are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the adaptor comprises a receptacle slot, which is configured to accommodate the QMAX device when the device is in a closed configuration. In certain embodiments, the QMAX device has a sample deposited therein and the adaptor can be connected to a mobile device (e.g. a smartphone) so that the sample can be read by the mobile device. In certain embodiments, the mobile device can detect and/or analyze a signal from the sample. In certain embodiments, the mobile device can capture images of the sample when the sample is in the QMAX device and positioned in the field of view (FOV) of a camera, which in certain embodiments, is part of the mobile device.

In some embodiments, the adaptor comprises optical components, which are configured to enhance, magnify, and/or optimize the production of the signal from the sample. In some embodiments, the optical components include parts that are configured to enhance, magnify, and/or optimize illumination provided to the sample. In certain embodiments, the illumination is provided by a light source that is part of the mobile device. In some embodiments, the optical components include parts that are configured to enhance, magnify, and/or optimize a signal from the sample.

### (6) Smartphone Detection System

The devices/apparatus, systems, and methods herein disclosed can include or use Q-cards for sample detection, analysis, and quantification. In some embodiments, the Q-card is used together with an adaptor that can connect the Q-card with a smartphone detection system. In some embodiments, the smartphone comprises a camera and/or an illumination source The smartphone detection system, as well the associated hardware and software are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the smartphone comprises a camera, which can be used to capture images or the sample when the sample is positioned in the field of view of the camera (e.g. by an adaptor). In certain embodiments, the camera includes one set of lenses (e.g. as in iPhone™ 6). In certain embodiments, the camera includes at least two sets of lenses (e.g. as in iPhone™ 7). In some embodiments, the smartphone comprises a camera, but the camera is not used for image capturing.

In some embodiments, the smartphone comprises a light source such as but not limited to LED (light emitting diode). In certain embodiments, the light source is used to provide illumination to the sample when the sample is positioned in the field of view of the camera (e.g. by an adaptor). In some embodiments, the light from the light source is enhanced, magnified, altered, and/or optimized by optical components of the adaptor.

In some embodiments, the smartphone comprises a processor that is configured to process the information from the sample. The smartphone includes software instructions that, when executed by the processor, can enhance, magnify, and/or optimize the signals (e.g. images) from the sample. The processor can include one or more hardware components, such as a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

In some embodiments, the smartphone comprises a communication unit, which is configured and/or used to transmit data and/or images related to the sample to another device. Merely by way of example, the communication unit can use a cable network, a wireline network, an optical fiber network, a telecommunications network, an intranet, the Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof.

In some embodiments, the smartphone is an iPhone™, an Android™ phone, or a Windows™ phone.

### (7) Detection Methods

The devices/apparatus, systems, and methods herein disclosed can include or be used in various types of detection methods. The detection methods are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287, 62/456,528, 62/456631, 62/456522, 62/456598, 62/456603, and 62/456,628, which were filed on Feb. 8, 2017, U.S. Provisional Application Nos. 62/459,276, 62/456,904, 62/457075, and 62/457,009, which were filed on Feb. 9, 2017, and U.S. Provisional Application Nos. 62/459,303, 62/459,337, and 62/459,598, which were filed on Feb. 15, 2017, and U.S. Provisional Application Nos. 62/460,083, 62/460,076, which were filed on Feb. 16, 2017, all of which applications are incorporated herein in their entireties for all purposes.

### (8) Labels, Capture Agent and Detection Agent

The devices/apparatus, systems, and methods herein disclosed can employ various types of labels, capture agents, and detection agents that are used for analytes detection. The labels are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the label is optically detectable, such as but not limited to a fluorescence label. In some embodiments, the labels include, but are not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino- -fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like.

In any embodiment, the QMAX device can contain a plurality of capture agents and/or detection agents that each bind to a biomarker selected from Tables B1, B2, B3 and/or B7 in U.S. Provisional Application No. 62/234,538 and/or PCT Application No. PCT/US2016/054025., wherein the reading step d) includes obtaining a measure of the amount of the plurality of biomarkers in the sample, and wherein the amount of the plurality of biomarkers in the sample is diagnostic of a disease or condition.

In any embodiment, the capture agent and/or detection agents can be an antibody epitope and the biomarker can be an antibody that binds to the antibody epitope. In some embodiments, the antibody epitope includes a biomolecule, or a fragment thereof, selected from Tables B4, B5 or B6 in U.S. Provisional Application No. 62/234,538 and/or PCT Application No. PCT/US2016/054025. In some embodiments, the antibody epitope includes an allergen, or a fragment thereof, selected from Table B5. In some embodiments, the antibody epitope includes an infectious agent-derived biomolecule, or a fragment thereof, selected from Table B6 in U.S. Provisional Application No. 62/234,538 and/or PCT Application No. PCT/US2016/054025.

In any embodiment, the QMAX device can contain a plurality of antibody epitopes selected from Tables B4, B5 and/or B6 in U.S. Provisional Application No. 62/234,538 and/or PCT Application No. PCT/US2016/054025, wherein the reading step d) includes obtaining a measure of the amount of a plurality of epitope-binding antibodies in the sample, and wherein the amount of the plurality of epitope-binding antibodies in the sample is diagnostic of a disease or condition.

### (9) Analytes

The devices/apparatus, systems, and methods herein disclosed can be applied to manipulation and detection of various types of analytes (including biomarkers). The analytes are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for the detection, purification and/or quantification of various analytes. In some embodiments, the analytes are biomarkers that associated with various diseases. In some embodiments, the analytes and/or biomarkers are indicative of the presence, severity, and/or stage of the diseases. The analytes, biomarkers, and/or diseases that can be detected and/or measured with the devices, apparatus, systems, and/or method of the present invention include the analytes, biomarkers, and/or diseases listed, described and/or summarized in PCT Application (designating U.S.) Nos. PCT/US2016/046437 filed on Aug. 10, 2016, and PCT Application No. PCT/US2016/054025 filed on Sep. 27, 2016, and U.S. Provisional Application Nos. 62/234,538 filed on Sep. 29, 2015, 62/233,885 filed on Sep. 28, 2015, 62/293,188 filed on Feb. 9, 2016, and 62/305,123 filed on Mar. 8, 2016, which are all hereby incorporated by reference by their entireties. For example, the devices, apparatus, systems, and methods herein disclosed can be used in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the analyte can be a biomarker, an environmental marker, or a foodstuff marker. The sample in some instances is a liquid sample, and can be a diagnostic sample (such as saliva, serum, blood, sputum, urine, sweat, lacrima, semen, or mucus); an environmental sample obtained from a river, ocean, lake, rain, snow, sewage, sewage processing runoff, agricultural runoff, industrial runoff, tap water or drinking water; or a foodstuff sample obtained from tap water, drinking water, prepared food, processed food or raw food.

In any embodiment, the sample can be a diagnostic sample obtained from a subject, the analyte can be a biomarker, and the measured the amount of the analyte in the sample can be diagnostic of a disease or a condition.

In any embodiment, the devices, apparatus, systems, and methods in the present invention can further include diagnosing the subject based on information including the measured amount of the biomarker in the sample. In some cases, the diagnosing step includes sending data containing the measured amount of the biomarker to a remote location and receiving a diagnosis based on information including the measurement from the remote location.

In any embodiment, the biomarker can be selected from Tables B1, 2, 3 or 7 as disclosed in U.S. Provisional Application Nos. 62/234,538, 62/293,188, and/or 62/305,123, and/or PCT Application No. PCT/US2016/054025, which are all incorporated in their entireties for all purposes. In some instances, the biomarker is a protein selected from Tables B1, 2, or 3. In some instances, the biomarker is a nucleic acid selected from Tables B2, 3 or 7. In some instances, the biomarker is an infectious agent-derived biomarker selected from Table B2. In some instances, the biomarker is a microRNA (miRNA) selected from Table B7.

In any embodiment, the applying step b) can include isolating miRNA from the sample to generate an isolated miRNA sample, and applying the isolated miRNA sample to the disk-coupled dots-on-pillar antenna (QMAX device) array.

In any embodiment, the QMAX device can contain a plurality of capture agents that each bind to a biomarker selected from Tables B1, B2, B3 and/or B7, wherein the reading step d) includes obtaining a measure of the amount of the plurality of biomarkers in the sample, and wherein the amount of the plurality of biomarkers in the sample is diagnostic of a disease or condition.

In any embodiment, the capture agent can be an antibody epitope and the biomarker can be an antibody that binds to the antibody epitope. In some embodiments, the antibody epitope includes a biomolecule, or a fragment thereof, selected from Tables B4, B5 or B6. In some embodiments, the antibody epitope includes an allergen, or a fragment thereof, selected from Table B5. In some embodiments, the antibody epitope includes an infectious agent-derived biomolecule, or a fragment thereof, selected from Table B6.

In any embodiment, the QMAX device can contain a plurality of antibody epitopes selected from Tables B4, B5 and/or B6, wherein the reading step d) includes obtaining a measure of the amount of a plurality of epitope-binding antibodies in the sample, and wherein the amount of the plurality of epitope-binding antibodies in the sample is diagnostic of a disease or condition.

In any embodiment, the sample can be an environmental sample, and wherein the analyte can be an environmental marker. In some embodiments, the environmental marker is selected from Table B8 in U.S. Provisional Application No. 62/234,538 and/or PCT Application No. PCT/US2016/054025.

In any embodiment, the method can include receiving or providing a report that indicates the safety or harmfulness for a subject to be exposed to the environment from which the sample was obtained.

In any embodiment, the method can include sending data containing the measured amount of the environmental marker to a remote location and receiving a report that indicates the safety or harmfulness for a subject to be exposed to the environment from which the sample was obtained.

In any embodiment, the QMAX device array can include a plurality of capture agents that each binds to an environmental marker selected from Table B8, and wherein the reading step d) can include obtaining a measure of the amount of the plurality of environmental markers in the sample.

In any embodiment, the sample can be a foodstuff sample, wherein the analyte can be a foodstuff marker, and wherein the amount of the foodstuff marker in the sample can correlate with safety of the foodstuff for consumption. In some embodiments, the foodstuff marker is selected from Table B9.

In any embodiment, the method can include receiving or providing a report that indicates the safety or harmfulness for a subject to consume the foodstuff from which the sample is obtained.

In any embodiment, the method can include sending data containing the measured amount of the foodstuff marker to a remote location and receiving a report that indicates the safety or harmfulness for a subject to consume the foodstuff from which the sample is obtained.

In any embodiment, the devices, apparatus, systems, and methods herein disclosed can include a plurality of capture agents that each binds to a foodstuff marker selected from Table B9 from in U.S. Provisional Application No. 62/234,538 and PCT Application No. PCT/US2016/054025, wherein the obtaining can include obtaining a measure of the amount of the plurality of foodstuff markers in the sample, and wherein the amount of the plurality of foodstuff marker in the sample can correlate with safety of the foodstuff for consumption.

Also provided herein are kits that find use in practicing the devices, systems and methods in the present invention.

The amount of sample can be about a drop of a sample. The amount of sample can be the amount collected from a pricked finger or fingerstick. The amount of sample can be the amount collected from a microneedle or a venous draw.

A sample can be used without further processing after obtaining it from the source, or can be processed, e.g., to enrich for an analyte of interest, remove large particulate matter, dissolve or resuspend a solid sample, etc.

Any suitable method of applying a sample to the QMAX device can be employed. Suitable methods can include using a pipet, dropper, syringe, etc. In certain embodiments, when the QMAX device is located on a support in a dipstick format, as described below, the sample can be applied to the QMAX device by dipping a sample-receiving area of the dipstick into the sample.

A sample can be collected at one time, or at a plurality of times. Samples collected over time can be aggregated and/or processed (by applying to a QMAX device and obtaining a measurement of the amount of analyte in the sample, as described herein) individually. In some instances, measurements obtained over time can be aggregated and can be useful for longitudinal analysis over time to facilitate screening, diagnosis, treatment, and/or disease prevention.

Washing the QMAX device to remove unbound sample components can be done in any convenient manner, as described above. In certain embodiments, the surface of the QMAX device is washed using binding buffer to remove unbound sample components.

Detectable labeling of the analyte can be done by any convenient method. The analyte can be labeled directly or indirectly. In direct labeling, the analyte in the sample is labeled before the sample is applied to the QMAX device. In indirect labeling, an unlabeled analyte in a sample is labeled after the sample is applied to the QMAX device to capture the unlabeled analyte, as described below.

### (10) Applications

The devices/apparatus, systems, and methods herein disclosed can be used for various applications (fields and samples). The applications are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the devices, apparatus, systems, and methods herein disclosed are used in a variety of different application in various field, wherein determination of the presence or absence, quantification, and/or amplification of one or more analytes in a sample are desired. For example, in certain embodiments the subject devices, apparatus, systems, and methods are used in the detection of proteins, peptides, nucleic acids, synthetic compounds, inorganic compounds, organic compounds, bacteria, virus, cells, tissues, nanoparticles, and other molecules, compounds, mixtures and substances thereof. The various fields in which the subject devices, apparatus, systems, and methods can be used include, but are not limited to: diagnostics, management, and/or prevention of human diseases and conditions, diagnostics, management, and/or prevention of veterinary diseases and conditions, diagnostics, management, and/or prevention of plant diseases and conditions, agricultural uses, veterinary uses, food testing, environments testing and decontamination, drug testing and prevention, and others.

The applications of the present invention include, but are not limited to: (a) the detection, purification, quantification, and/or amplification of chemical compounds or biomolecules that correlates with certain diseases, or certain stages of the diseases, e.g., infectious and parasitic disease, injuries, cardiovascular disease, cancer, mental disorders, neuropsychiatric disorders and organic diseases, e.g., pulmonary diseases, renal diseases, (b) the detection, purification, quantification, and/or amplification of cells and/or microorganism, e.g., virus, fungus and bacteria from the environment, e.g., water, soil, or biological samples, e.g., tissues, bodily fluids, (c) the detection, quantification of chemical compounds or biological samples that pose hazard to food safety, human health, or national security, e.g. toxic waste, anthrax, (d) the detection and quantification of vital parameters in medical or physiological monitor, e.g., glucose, blood oxygen level, total blood count, (e) the detection and quantification of specific DNA or RNA from biological samples, e.g., cells, viruses, bodily fluids, (f) the sequencing and comparing of genetic sequences in DNA in the chromosomes and mitochondria for genome analysis or (g) the detection and quantification of reaction products, e.g., during synthesis or purification of pharmaceuticals.

In some embodiments, the subject devices, apparatus, systems, and methods are used in the detection of nucleic acids, proteins, or other molecules or compounds in a sample. In certain embodiments, the devices, apparatus, systems, and methods are used in the rapid, clinical detection and/or quantification of one or more, two or more, or three or more disease biomarkers in a biological sample, e.g., as being employed in the diagnosis, prevention, and/or management of a disease condition in a subject. In certain embodiments, the devices, apparatus, systems, and methods are used in the detection and/or quantification of one or more, two or more, or three or more environmental markers in an environmental sample, e.g. sample obtained from a river, ocean, lake, rain, snow, sewage, sewage processing runoff, agricultural runoff, industrial runoff, tap water or drinking water. In certain embodiments, the devices, apparatus, systems, and methods are used in the detection and/or quantification of one or more, two or more, or three or more foodstuff marks from a food sample obtained from tap water, drinking water, prepared food, processed food or raw food.

In some embodiments, the subject device is part of a microfluidic device. In some embodiments, the subject devices, apparatus, systems, and methods are used to detect a fluorescence or luminescence signal. In some embodiments, the subject devices, apparatus, systems, and methods include, or are used together with, a communication device, such as but not limited to: mobile phones, tablet computers and laptop computers. In some embodiments, the subject devices, apparatus, systems, and methods include, or are used together with, an identifier, such as but not limited to an optical barcode, a radio frequency ID tag, or combinations thereof.

In some embodiments, the sample is a diagnostic sample obtained from a subject, the analyte is a biomarker, and the measured amount of the analyte in the sample is diagnostic of a disease or a condition. In some embodiments, the subject devices, systems and methods further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In some embodiments, the sample is an environmental sample, and wherein the analyte is an environmental marker. In some embodiments, the subject devices, systems and methods includes receiving or providing a report that indicates the safety or harmfulness for a subject to be exposed to the environment from which the sample was obtained. In some embodiments, the subject devices, systems and methods include sending data containing the measured amount of the environmental marker to a remote location and receiving a report that indicates the safety or harmfulness for a subject to be exposed to the environment from which the sample was obtained.

In some embodiments, the sample is a foodstuff sample, wherein the analyte is a foodstuff marker, and wherein the amount of the foodstuff marker in the sample correlate with safety of the foodstuff for consumption. In some embodiments, the subject devices, systems and methods include receiving or providing a report that indicates the safety or harmfulness for a subject to consume the foodstuff from which the sample is obtained. In some embodiments, the subject devices, systems and methods include sending data containing the measured amount of the foodstuff marker to a remote location and receiving a report that indicates the safety or harmfulness for a subject to consume the foodstuff from which the sample is obtained.

### (11) Dimensions

The devices, apparatus, systems, and methods herein disclosed can include or use a QMAX device, which can comprise plates and spacers. In some embodiments, the dimension of the individual components of the QMAX device and its adaptor are listed, described and/or summarized in PCT Application (designating U.S.) No. PCT/US2016/046437 filed on Aug. 10, 2016, and U.S Provisional Application Nos. 62,431,639 filed on Dec. 9, 2016 and 62/456,287 filed on Feb. 8, 2017, which are all hereby incorporated by reference by their entireties.

In some embodiments, the dimensions are listed in the Tables below:

### Plates:

| | | Preferred |
|---|---|---|
| Parameters | Embodiments | Embodiments |
| Shape | round, ellipse, rectangle, | at least one of |
| | triangle, polygonal, | the two (or |
| | ring-shaped, or any | more) plates of the |
| | superposition of these | QMAX |
| | shapes; the two (or more) | card has round |
| | plates of the QMAX | corners for user safety |
| | card can have the same | concerns, wherein the |
| | size and/or shape, or | round corners have a |
| | different size and/or shape; | diameter of 100 um or |
| | | less, 200 um or less, |
| | | 500 um or less, 1 mm or |
| | | less, 2 mm or less, 5 mm |
| | | or less, 10 mm or less, 50 |
| | | mm or less, or in a range |
| | | between any two of the |
| | | values. |
| Thickness | the average thickness | For at least one of the |
| | for at least one of the | plates is in the range of |
| | plates is 2 nm or less, | 0.5 to 1.5 mm; around 1 |
| | 10 nm or less, 100 nm | mm; in the range of 0.15 |
| | or less, 200 nm or less, | to 0.2 mm; or around |
| | 500 nm or less, 1000 | 0.175 mm |
| | nm or less, 2 μm (micron) | |
| | or less, 5 μm or | |
| | less, 10 μm or less, | |
| | 20 μm or less, 50 μm or | |
| | less, 100 μm or less, | |
| | 150 μm or less, 200 μm | |
| | or less, 300 μm or less, | |
| | 500 μm or less, 800 | |
| | μm or less, 1 mm | |
| | (millimeter) or less, 2 mm or | |
| | less, 3 mm or less, | |
| | 5 mm or less, 10 mm or | |
| | less, 20 mm or less, | |
| | 50 mm or less, 100 mm or | |
| | less, 500 mm or less, | |
| | or in a range between | |
| | any two of these values | |
| Lateral | For at least one of the | For at least one plate of |
| Area | plate is 1 mm2 (square | the QMAX |
| | millimeter) or less, | card is in the |
| | 10 mm2 or less, 25 mm2 or | range of 500 to 1000 |
| | less, 50 mm2 or less, | mm²; or around 750 mm². |
| | 75 mm2 or less, 1 cm2 | |
| | (square centimeter) | |
| | or less, 2 cm2 or less, 3 | |
| | cm2 or less, 4 cm2 or | |
| | less, 5 cm2 or less, 10 | |
| | cm2 or less, 100 cm2 | |
| | or less, 500 cm2 or less, | |
| | 1000 cm2 or less, 5000 | |
| | cm2 or less, 10,000 | |
| | cm2 or less, 10,000 | |
| | cm2 or less, or in a range | |
| | between any two | |
| | of these values | |
| Lateral | For at least one of the | For at least one plate of |
| Linear | plates of the QMAX card | QMAX card is in the |
| Dimension | is 1 mm or less, 5 mm or | range of 20 to 30 mm; or |
| (width, | less, 10 mm or less, the | around 24 mm |
| length, or | 15 mm or less, 20 mm | |
| diameter, | or less, 25 mm or less, | |
| etc.) | 30 mm or less, 35 mm | |
| | or less, 40 mm or less, | |
| | 45 mm or less, 50 mm | |
| | or less, 100 mm or less, | |
| | 200 mm or less, 500 mm | |
| | or less, 1000 mm or | |
| | less, 5000 mm or less, | |
| | or in a range between | |
| | any two of these values | |
| Recess | 1 μm or less, 10 μm | In the range of 1 mm to |
| width | or less, 20 μm or less, 30 | 10 mm; Or |
| | μm or less, 40 μm or | About 5 mm |
| | less, 50 μm or less, 100 | |
| | μm or less, 200 μm or | |
| | less, 300 μm or less, | |
| | 400 μm or less, 500 μm | |
| | or less, 7500 μm or | |
| | less, 1 mm or less, | |
| | 5 mm or less, 10 mm or | |
| | less, 100 mm or less, | |
| | or 1000 mm or less, or in | |
| | a range between any | |
| | two of these values. | |

### Hinge:

| | | Preferred |
|---|---|---|
| Parameters | Embodiments | Embodiments |
| Number | 1, 2, 3, 4, 5, or more | 1 or 2 |
| Length of | 1 mm or less, 2 mm | In the range |
| Hinge | or less, 3 mm or less, 4 | of 5 mm to |
| Joint | mm or less, 5 mm or | 30 mm. |
| | less, 10 mm or less, 15 | |
| | mm or less, 20 mm | |
| | or less, 25 mm or less, | |
| | 30 mm or less, | |
| | 40 mm or less, 50 mm or | |
| | less, 100 mm or less, | |
| | 200 mm or less, or | |
| | 500 mm or less, or in | |
| | a range between any | |
| | two of these values | |
| Ratio | 1.5 or less, 1 or less, | In the range |
| (hinge | 0.9 or less, 0.8 or less, | of 0.2 to 1; or |
| joint | 0.7 or less, 0.6 or less, | about 1 |
| length | 0.5 or less, 0.4 or | |
| vs. | less, 0.3 or less, 0.2 | |
| aligning | or less, 0.1 or less, 0.05 | |
| plate | or less or in a range | |
| edge | between any two of | |
| length | these values. | |
| Area | 1 mm² or less, 5 mm² | In the range |
| | or less, 10 mm² or less, | of 20 to 200 |
| | 20 mm² or less, 30 | mm²; or |
| | mm² or less, 40 mm² or | about 120 mm² |
| | less, 50 mm² or less, | |
| | 100 mm² or less, 200 | |
| | mm² or less, 500 mm² | |
| | or less, or in a range | |
| | between any of the | |
| | two values | |
| Ratio | 1 or less, 0.9 or less, | In the range |
| (hinge | 0.8 or less, 0.7 or less, | of 0.05 to |
| area vs. | 0.6 or less, 0.5 or less, | 0.2, around 0.15 |
| plate | 0.4 or less, 0.3 or less, | |
| area) | 0.2 or less, 0.1 or less, | |
| | 0.05 or less, 0.01 or | |
| | less or in a range | |
| | between any two of these | |
| | values | |
| Max. | 15 or less, 30 or less, | In the range |
| Open | 45 or less, 60 or less, | of 90 to 180 |
| Degree | 75 or less, 90 or less, | degrees |
| | 105 or less, 120 or less, | |
| | 135 or less, 150 or less, | |
| | 165 or less, 180 or | |
| | less, 195 or less, 210 | |
| | or less, 225 or less, 240 | |
| | or less, 255 or less, | |
| | 270 or less, 285 or less, | |
| | 300 or less, 315 or | |
| | less, 330 or less, 345 or | |
| | less or 360 or less | |
| | degrees, or in a range | |
| | between any two | |
| | of these values | |
| No. of | 1, 2, 3, 4, 5, or more | 1 or 2 |
| Layers | | |
| Layer | 0.1 μm or less, 1 μm | In the range |
| thickness | or less, 2 μm or less, 3 | of 20 μm to |
| | μm or less, 5 μm or less, | 1 mm; or |
| | 10 μm or less, 20 | Around 50 μm |
| | μm or less, 30 μm or | |
| | less, 50 μm or less, 100 | |
| | μm or less, 200 μm or | |
| | less, 300 μm or less, | |
| | 500 μm or less, 1 mm | |
| | or less, 2 mm or less, | |
| | and a range between | |
| | any two of these values | |
| Angle- | Limiting the angle | No more than ±2 |
| maintaining | adjustment with no more | |
| | than ±90, ±45, ±30, | |
| | ±25, ±20, ±15, ±10, ±8, | |
| | ±6, ±5, ±4, ±3, ±2, | |
| | or ±1, or in a range | |
| | between any two of | |
| | these values | |

### Notch:

| | | Preferred |
|---|---|---|
| Parameters | Embodiments | Embodiments |
| Number | 1, 2, 3, 4, 5, or more | 1 or 2 |
| Shape | round, ellipse, rectangle, | Part of a circle |
| | triangle, polygon, | |
| | ring-shaped, or any | |
| | superposition or portion | |
| | of these shapes. | |
| Positioning | Any location along | |
| | any edge except the | |
| | hinge edge, or any | |
| | corner joint by non-hinge | |
| | edges | |
| Lateral | 1 mm or less, 2.5 mm | In the range |
| Linear | or less, 5 mm or less, | of 5 mm to |
| Dimension | 10 mm or less, 15 mm | 15 mm; or |
| (Length | or less, 20 mm or | about 10 mm |
| along the | less, 25 mm or less, | |
| edge, | 30 mm or less, 40 mm | |
| radius, | or less, 50 mm or | |
| etc.) | less, or in a range | |
| | between any two | |
| | of these values | |
| Area | 1 mm² (square millimeter) | In the range |
| | or less, 10 mm² or | of 10 to 150 |
| | less, 25 mm² or less, | mm²; or |
| | 50 mm² or less, 75 mm² | about 50 mm² |
| | or less or in a range | |
| | between any two of these | |
| | values. | |

### Trench:

| | | Preferred |
|---|---|---|
| Parameters | Embodiments | Embodiments |
| Number | 1, 2, 3, 4, 5, or more | 1 or 2 |
| Shape | Closed (round, ellipse, | |
| | rectangle, triangle, | |
| | polygon, ring-shaped, | |
| | or any superposition or | |
| | portion of these shapes) | |
| | or open-ended | |
| | (straight line, curved | |
| | line, arc, branched tree, | |
| | or any other shape | |
| | with open endings); | |
| Length | 0.001 mm or less, | |
| | 0.005 mm or less, 0.01 | |
| | mm or less, 0.05 mm | |
| | or less, 0.1 mm or less, | |
| | 0.5 mm or less, 1 mm | |
| | or less, 2 mm or less, | |
| | 5 mm or less, 10 mm | |
| | or less, 20 mm or less, | |
| | 50 mm or less, 100 mm | |
| | or less, or in a range | |
| | between any two of | |
| | these values | |
| Cross- | 0.001 mm² or less, | |
| sectional | 0.005 mm² or less, 0.01 | |
| Area | mm² or less, 0.05 mm² | |
| | or less, 0.1 mm² or | |
| | less, 0.5 mm² or less, | |
| | 1 mm² or less, 2 mm² or | |
| | less, 5 mm² or less, 10 | |
| | mm² or less, 20 mm² | |
| | or less, or in a range | |
| | between any two of these | |
| | values. | |
| Volume | 0.1 μL or more, 0.5 μL or | In the range |
| | more, 1 μL or more, | of 1 μL to |
| | 2 μL or more, 5 μL or | 20 μL; or |
| | more, 10 μL or more, 30 | About 5 μL |
| | μL or more, 50 μL | |
| | or more, 100 μL or more, | |
| | 500 μL or more, 1 mL | |
| | or more, or in a range | |
| | between any two of | |
| | these values | |

### Receptacle Slot

| | | Preferred |
|---|---|---|
| Parameters | Embodiments | Embodiments |
| Shape of | round, ellipse, rectangle, | |
| receiving | triangle, polygon, | |
| area | ring-shaped, or any | |
| | superposition of these | |
| | shapes; | |
| Difference | 100 nm, 500 nm, 1 μm, | In the range |
| between | 2 μm, 5 μm, 10 μm, 50 | of 50 to 300 |
| sliding track | μm, 100 μm, 300 μm, | μm; or about |
| gap size | 500 μm, 1 mm, 2 mm, | 75 μm |
| and card | 5 mm, 1 cm, or in a | |
| thickness | range between any two | |
| | of the values. | |
| Difference | 1 mm² (square millimeter) | |
| between | or less, 10 mm² or | |
| receiving | less, 25 mm² or less, 50 | |
| area and | mm² or less, 75 mm² | |
| card area | or less, 1 cm² (square | |
| | centimeter) or less, 2 | |
| | cm² or less, 3 cm² or | |
| | less, 4 cm² or less, 5 cm² | |
| | or less, 10 cm² or less, | |
| | 100 cm² or less, or in | |
| | a range between any | |
| | of the two values. | |

### (12) Cloud

The devices/apparatus, systems, and methods herein disclosed can employ cloud technology for data transfer, storage, and/or analysis. The related cloud technologies are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, US Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the cloud storage and computing technologies can involve a cloud database. Merely by way of example, the cloud platform can include a private cloud, a public cloud, a hybrid cloud, a community cloud, a distributed cloud, an inter-cloud, a multi-cloud, or the like, or any combination thereof. In some embodiments, the mobile device (e.g. smartphone) can be connected to the cloud through any type of network, including a local area network (LAN) or a wide area network (WAN).

In some embodiments, the data (e.g. images of the sample) related to the sample is sent to the cloud without processing by the mobile device and further analysis can be conducted remotely. In some embodiments, the data related to the sample is processed by the mobile device and the results are sent to the cloud. In some embodiments, both the raw data and the results are transmitted to the cloud.

### Flat Top of Pillar Spacers

In certain embodiments of the present invention, the spacers are pillars that have a flat top and a foot fixed on one plate, wherein the flat top has a smoothness with a small surface variation, and the variation is less than 5, 10 nm, 20 nm, 30 nm, 50 nm, 100 nm, 200 nm, 300 nm, 400 nm, 500 nm, 600 nm, 700 nm, 800 nm, 1000 nm, or in a range between any two of the values. A preferred flat pillar top smoothness is that surface variation of 50 nm or less.

Furthermore, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%.

### Sidewall Angle of Pillar Spacers

In certain embodiments of the present invention, the spacers are pillars that have a sidewall angle. In some embodiments, the sidewall angle is less than 5 degree (measured from the normal of a surface), 10 degree, 20 degree, 30 degree, 40 degree, 50 degree, 70 degree, or in a range between any two of the values. In a preferred embodiment, the sidewall angle is less 5 degree, 10 degree, or 20 degree.

### Formation of Uniform Thin Fluidic Layer by an Imprecise Force Pressing

In certain embodiment of the present invention, a uniform thin fluidic sample layer is formed by using a pressing with an imprecise force. The term “imprecise pressing force” without adding the details and then adding a definition for imprecise pressing force. As used herein, the term “imprecise” in the context of a force (e.g. “imprecise pressing force”) refers to a force that

(a) has a magnitude that is not precisely known or precisely predictable at the time the force is applied; (b) has a pressure in the range of 0.01 kg/cm² (centimeter square) to 100 kg/cm², (c) varies in magnitude from one application of the force to the next; and (d) the imprecision (i.e. the variation) of the force in (a) and (c) is at least 20% of the total force that actually is applied.

An imprecise force can be applied by human hand, for example, e.g., by pinching an object together between a thumb and index finger, or by pinching and rubbing an object together between a thumb and index finger.

In some embodiments, the imprecise force by the hand pressing has a pressure of 0.01 kg/cm2, 0.1 kg/cm2, 0.5 kg/cm2, 1 kg/cm2, 2 kg/cm2, kg/cm2, 5 kg/cm2, 10 kg/cm2, 20 kg/cm2, 30 kg/cm2, 40 kg/cm2, 50 kg/cm2, 60 kg/cm2, 100 kg/cm2, 150 kg/cm2, 200 kg/cm2, or a range between any two of the values; and a preferred range of 0.1 kg/cm2 to 0.5 kg/cm2, 0.5 kg/cm2 to 1 kg/cm2, 1 kg/cm2 to 5 kg/cm2, 5 kg/cm2 to 10 kg/cm2 (Pressure).

### Spacer Filling Factor.

The term “spacer filling factor” or “filling factor” refers to the ratio of the spacer contact area to the total plate area”, wherein the spacer contact area refers, at a closed configuration, the contact area that the spacer's top surface contacts to the inner surface of a plate, and the total plate area refers the total area of the inner surface of the plate that the flat top of the spacers contact. Since there are two plates and each spacer has two contact surfaces each contacting one plate, the filling fact is the filling factor of the smallest.

For example, if the spacers are pillars with a flat top of a square shape (10 μm×10 μm), a nearly uniform cross-section and 2 μm tall, and the spacers are periodic with a period of 100 μm, then the filing factor of the spacer is 1%. If in the above example, the foot of the pillar spacer is a square shape of 15 μm×15 μm, then the filling factor is still 1% by the definition.

### IDS\hat{ }4/hE

In certain embodiments of the present disclosure, a device for forming a thin fluidic sample layer with a uniform predetermined thickness by pressing can comprise a first plate. In certain embodiments of the present disclosure, a device for forming a thin fluidic sample layer with a uniform predetermined thickness by pressing can comprise a second plate. In certain embodiments of the present disclosure, a device for forming a thin fluidic sample layer with a uniform predetermined thickness by pressing can comprise spacers. In certain embodiments, the plates are movable relative to each other into different configurations. In certain embodiments, one or both plates are flexible. In certain embodiments, each of the plates comprises an inner surface that has a sample contact area for contacting a fluidic sample. In certain embodiments, each of the plates comprises, on its respective outer surface, a force area for applying a pressing force that forces the plates together. In certain embodiments, one or both of the plates comprise the spacers that are permanently fixed on the inner surface of a respective plate. In certain embodiments, the spacers have a predetermined substantially uniform height that is equal to or less than 200 microns, and a predetermined fixed inter-spacer-distance. In certain embodiments, the fourth power of the inter-spacer-distance (ISD) divided by the thickness (h) and the Young's modulus (E) of the flexible plate (ISD⁴/(hE)) is 5×10⁶ um³/GPa or less. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample is deposited in the open configuration and the plates are forced to the closed configuration by applying the pressing force on the force area; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure, a method of forming a thin fluidic sample layer with a uniform predetermined thickness by pressing can comprise depositing a fluidic sample on one or both of the plates when the plates are configured in an open configuration. In certain embodiments, the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for analyzing a fluidic sample can comprise a first plate. In certain embodiments of the present disclosure, a device for analyzing a fluidic sample can comprise a second plate. In certain embodiments of the present disclosure, a device for analyzing a fluidic sample can comprise spacers. In certain embodiments, the plates are movable relative to each other into different configurations. In certain embodiments, one or both plates are flexible. In certain embodiments, each of the plates has, on its respective inner surface, a sample contact area for contacting a fluidic sample. In certain embodiments, one or both of the plates comprise the spacers and the spacers are fixed on the inner surface of a respective plate. In certain embodiments, the spacers have a predetermined substantially uniform height that is equal to or less than 200 microns, and the inter-spacer-distance is predetermined. In certain embodiments, the Young's modulus of the spacers multiplied by the filling factor of the spacers is at least 2 MPa. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample is deposited in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure, a method of forming a thin fluidic sample layer with a uniform predetermined thickness by pressing can comprise depositing a fluidic sample on one or both of the plates when the plates are configured in an open configuration. In certain embodiments, the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing can comprise forcing the two plates into a closed configuration. In certain embodiments, at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for analyzing a fluidic sample can comprise a first plate. In certain embodiments of the present disclosure, a device for analyzing a fluidic sample can comprise a second plate. In certain embodiments, the plates are movable relative to each other into different configurations. In certain embodiments, one or both plates are flexible. In certain embodiments, each of the plates has, on its respective surface, a sample contact area for contacting a sample that contains an analyte. In certain embodiments, one or both of the plates comprise spacers that are permanently fixed to a plate within a sample contact area, wherein the spacers have a predetermined substantially uniform height and a predetermined fixed inter-spacer distance that is at least about 2 times larger than the size of the analyte, up to 200 um, and wherein at least one of the spacers is inside the sample contact area. In certain embodiments, one of the configurations is an open configuration, in which: the two plates are separated apart, the spacing between the plates is not regulated by the spacers, and the sample is deposited on one or both of the plates. In certain embodiments, another of the configurations is a closed configuration which is configured after the sample deposition in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure a method of forming a thin fluidic sample layer with a uniform predetermined thickness by pressing can comprise depositing a fluidic sample on one or both of the plates; when the plates are configured in an open configuration, wherein the open configuration is a configuration in which the two plates are partially or completely separated apart and the spacing between the plates is not regulated by the spacers. In certain embodiments of the present disclosure a method of forming a thin fluidic sample layer with a uniform predetermined thickness by pressing can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for forming a thin fluidic sample layer with a uniform predetermined thickness by pressing can comprise a first plate. In certain embodiments of the present disclosure, a device for forming a thin fluidic sample layer with a uniform predetermined thickness by pressing can comprise a second plate. In certain embodiments of the present disclosure, a device for forming a thin fluidic sample layer with a uniform predetermined thickness by pressing can comprise spacers. In certain embodiments, the plates are movable relative to each other into different configurations. In certain embodiments, one or both plates are flexible. In certain embodiments, each of the plates comprises, on its respective inner surface, a sample contact area for contacting and/or compressing a fluidic sample. In certain embodiments, each of the plates comprises, on its respective outer surface, an area for applying a force that forces the plates together. In certain embodiments, one or both of the plates comprise the spacers that are permanently fixed on the inner surface of a respective plate. In certain embodiments, the spacers have a predetermined substantially uniform height that is equal to or less than 200 microns, a predetermined width, and a predetermined fixed inter-spacer-distance. In certain embodiments, a ratio of the inter-spacer-distance to the spacer width is 1.5 or larger. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments of the present disclosure, a method of forming a thin fluidic sample layer with a uniform predetermined thickness by pressing with an imprecise pressing force can comprise obtaining a device of the present disclosure. In certain embodiments of the present disclosure, a method of forming a thin fluidic sample layer with a uniform predetermined thickness by pressing with an imprecise pressing force can comprise obtaining a fluidic sample. In certain embodiments of the present disclosure, a method of forming a thin fluidic sample layer with a uniform predetermined thickness by pressing with an imprecise pressing force can comprise depositing the sample on one or both of the plates; when the plates are configured in an open configuration, wherein the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing with an imprecise pressing force can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments, the spacers have a shape of pillar with a foot fixed on one of the plates and a flat top surface for contacting the other plate. In certain embodiments, the spacers have a shape of pillar with a foot fixed on one of the plates, a flat top surface for contacting the other plate, substantially uniform cross-section. In certain embodiments, the spacers have a shape of pillar with a foot fixed on one of the plates and a flat top surface for contacting the other plate, wherein the flat top surface of the pillars has a variation in less than 10 nm. In certain embodiments, the spacers have a shape of pillar with a foot fixed on one of the plates and a flat top surface for contacting the other plate, wherein the flat top surface of the pillars has a variation in less than 50 nm. In certain embodiments, the spacers have a shape of pillar with a foot fixed on one of the plates and a flat top surface for contacting the other plate, wherein the flat top surface of the pillars has a variation in less than 50 nm. In certain embodiments, the spacers have a shape of pillar with a foot fixed on one of the plates and a flat top surface for contacting the other plate, wherein the flat top surface of the pillars has a variation in less than 10 nm, 20 nm, 30 nm, 100 nm, 200 nm, or in a range of any two of the values.

In certain embodiments, the Young's modulus of the spacers multiplied by the filling factor of the spacers is at least 2 MPa. In certain embodiments, the sample comprises an analyte and the predetermined constant inter-spacer distance is at least about 2 times larger than the size of the analyte, up to 200 um. In certain embodiments, the sample comprise an analyte, the predetermined constant inter-spacer distance is at least about 2 times larger than the size of the analyte, up to 200 um, and the Young's modulus of the spacers multiplied by the filling factor of the spacers is at least 2 MPa.

In certain embodiments, a fourth power of the inter-spacer-distance (IDS) divided by the thickness (h) and the Young's modulus (E) of the flexible plate (ISD\hat{ }4/(hE)) is 5×10\hat{ }6 um\hat{ }3/GPa or less. In certain embodiments, a fourth power of the inter-spacer-distance (IDS) divided by the thickness and the Young's modulus of the flexible plate (ISD\hat{ }4/(hE)) is 1×10\hat{ }6 um\hat{ }3/GPa or less. In certain embodiments, a fourth power of the inter-spacer-distance (IDS) divided by the thickness and the Young's modulus of the flexible plate (ISD\hat{ }4/(hE)) is 5×10\hat{ }5 um\hat{ }3/GPa or less. In certain embodiments, the Young's modulus of the spacers multiplied by the filling factor of the spacers is at least 2 MPa, and a fourth power of the inter-spacer-distance (IDS) divided by the thickness and the Young's modulus of the flexible plate (ISD\hat{ }4/(hE)) is 1×10\hat{ }5 um\hat{ }3/GPa or less. In certain embodiments, the Young's modulus of the spacers multiplied by the filling factor of the spacers is at least 2 MPa, and a fourth power of the inter-spacer-distance (IDS) divided by the thickness and the Young's modulus of the flexible plate (ISD\hat{ }4/(hE)) is 1×10\hat{ }4 um\hat{ }3/GPa or less. In certain embodiments, the Young's modulus of the spacers multiplied by the filling factor of the spacers is at least 20 MPa.

In certain embodiments of the present disclosure, the ratio of the inter-spacing distance of the spacers to the average width of the spacer is 2 or larger. In certain embodiments, the ratio of the inter-spacing distance of the spacers to the average width of the spacer is 2 or larger, and the Young's modulus of the spacers multiplied by the filling factor of the spacers is at least 2 MPa. In certain embodiments, the inter-spacer distance that is at least about 2 times larger than the size of the analyte, up to 200 um. In certain embodiments, a ratio of the inter-spacer-distance to the spacer width is 1.5 or larger. In certain embodiments, a ratio of the width to the height of the spacer is 1 or larger. In certain embodiments, a ratio of the width to the height of the spacer is 1.5 or larger. In certain embodiments, a ratio of the width to the height of the spacer is 2 or larger. In certain embodiments, a ratio of the width to the height of the spacer is larger than 2, 3, 5, 10, 20, 30, 50, or in a range of any two the value.

In certain embodiments, a force that presses the two plates into the closed configuration is an imprecise pressing force. In certain embodiments, a force that presses the two plates into the closed configuration is an imprecise pressing force provided by human hand. In certain embodiments, the forcing of the two plates to compress at least part of the sample into a layer of substantially uniform thickness comprises a use of a conformable pressing, either in parallel or sequentially, an area of at least one of the plates to press the plates together to a closed configuration, wherein the conformable pressing generates a substantially uniform pressure on the plates over the at least part of the sample, and the pressing spreads the at least part of the sample laterally between the sample contact surfaces of the plates, and wherein the closed configuration is a configuration in which the spacing between the plates in the layer of uniform thickness region is regulated by the spacers; and wherein the reduced thickness of the sample reduces the time for mixing the reagents on the storage site with the sample. In certain embodiments, the pressing force is an imprecise force that has a magnitude which is, at the time that the force is applied, either (a) unknown and unpredictable, or (b) cannot be known and cannot be predicted within an accuracy equal or better than 20% of the average pressing force applied. In certain embodiments, the pressing force is an imprecise force that has a magnitude which is, at the time that the force is applied, either (a) unknown and unpredictable, or (b) cannot be known and cannot be predicted within an accuracy equal or better than 30% of the average pressing force applied. In certain embodiments, the pressing force is an imprecise force that has a magnitude which is, at the time that the force is applied, either (a) unknown and unpredictable, or (b) cannot be known and cannot be predicted within an accuracy equal or better than 30% of the average pressing force applied; and wherein the layer of highly uniform thickness has a variation in thickness uniform of 20% or less. In certain embodiments, the pressing force is an imprecise force that has a magnitude which cannot, at the time that the force is applied, be determined within an accuracy equal or better than 30%, 40%, 50%, 70%, 100%, 200%, 300%, 500%, 1000%, 2000%, or in a range between any of the two values.

In certain embodiments of the present disclosure, the flexible plate has a thickness of in the range of 10 um to 200 um. In certain embodiments, the flexible plate has a thickness of in the range of 20 um to 100 um. In certain embodiments, the flexible plate has a thickness of in the range of 25 urn to 180 urn. In certain embodiments, the flexible plate has a thickness of in the range of 200 urn to 260 urn. In certain embodiments, the flexible plate has a thickness of equal to or less than 250 um, 225 um, 200 um, 175 um, 150 um, 125 um, 100 um, 75 um, 50 um, 25 um, 10 um, 5 um, 1 um, or in a range between the two of the values. In certain embodiments, the sample has a viscosity in the range of 0.1 to 4 (mPa s). In certain embodiments, the flexible plate has a thickness of in the range of 200 urn to 260 urn. In certain embodiments, the flexible plate has a thickness in the range of 20 urn to 200 urn and Young's modulus in the range 0.1 to 5 GPa.

In certain embodiments of the present disclosure, the sample deposition is a deposition directly from a subject to the plate without using any transferring devices. In certain embodiments, during the deposition, the amount of the sample deposited on the plate is unknown. In certain embodiments, the method further comprises an analyzing that analyze the sample. In certain embodiments, the analyzing comprises calculating the volume of a relevant sample volume by measuring the lateral area of the relevant sample volume and calculating the volume from the lateral area and the predetermined spacer height. In certain embodiments, the pH value at location of a sample that is between the two plates in a closed configuration is determined by the volume of the location and by analyzing an image(s) taken from that location. In certain embodiments, the determination by analyzing an image uses artificial intelligence and machine learning.

In certain embodiments, the analyzing step (e) comprises measuring: i. imaging, ii. luminescence selected from photoluminescence, electroluminescence, and electrochemiluminescence, iii. surface Raman scattering, iv. electrical impedance selected from resistance, capacitance, and inductance, or v. any combination of i-iv. In certain embodiments, the analyzing comprises reading, image analysis, or counting of the analyte, or a combination of thereof. In certain embodiments, the sample contains one or plurality of analytes, and one or both plate sample contact surfaces comprise one or a plurality of binding sites that each binds and immobilize a respective analyte. In certain embodiments, one or both plate sample contact surfaces comprise one or a plurality of storage sites that each stores a reagent or reagents, wherein the reagent(s) dissolve and diffuse in the sample. In certain embodiments, one or both plate sample contact surfaces comprises one or a plurality of amplification sites that are each capable of amplifying a signal from the analyte or a label of the analyte when the analyte or label is within 500 nm from an amplification site. In certain embodiments, i. one or both plate sample contact surfaces comprise one or a plurality of binding sites that each binds and immobilize a respective analyte; or ii. one or both plate sample contact surfaces comprise, one or a plurality of storage sites that each stores a reagent or reagents; wherein the reagent(s) dissolve and diffuse in the sample, and wherein the sample contains one or plurality of analytes; or iii. one or a plurality of amplification sites that are each capable of amplifying a signal from the analyte or a label of the analyte when the analyte or label is 500 nm from the amplification site; or iv. any combination of i to iii.

In certain embodiments, the liquid sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

In certain embodiments, the layer of uniform thickness in the closed configuration is less than 150 um. In certain embodiments, the pressing is provided by a pressured liquid, a pressed gas, or a conformal material. In certain embodiments, the analyzing comprises counting cells in the layer of uniform thickness. In certain embodiments, the analyzing comprises performing an assay in the layer of uniform thickness. In certain embodiments, In certain embodiments, the assay is a binding assay or biochemical assay. In certain embodiments, the sample deposited has a total volume less 0.5 uL. In certain embodiments, multiple drops of sample are deposited onto one or both of the plates.

In certain embodiments, the inter-spacer distance is in the range of 1 μm to 120 μm. In certain embodiments, the inter-spacer distance is in the range of 120 μm to 50 μm. In certain embodiments, the inter-spacer distance is in the range of 120 μm to 200 μm. In certain embodiments, the flexible plates have a thickness in the range of 20 μm to 250 μm and Young's modulus in the range 0.1 to 5 GPa. In certain embodiments, for a flexible plate, the thickness of the flexible plate times the Young's modulus of the flexible plate is in the range 60 to 750 GPa-um.

In certain embodiments, the layer of uniform thickness sample is uniform over a lateral area that is at least 1 mm². In certain embodiments, the layer of uniform thickness sample is uniform over a lateral area that is at least 3 mm². In certain embodiments, the layer of uniform thickness sample is uniform over a lateral area that is at least 5 mm². In certain embodiments, In certain embodiments, the layer of uniform thickness sample is uniform over a lateral area that is at least 10 mm². In certain embodiments, the layer of uniform thickness sample is uniform over a lateral area that is at least 20 mm². In certain embodiments, the layer of uniform thickness sample is uniform over a lateral area that is in a range of 20 mm² to 100 mm². In certain embodiments, the layer of uniform thickness sample has a thickness uniformity of up to +/−5% or better. In certain embodiments, the layer of uniform thickness sample has a thickness uniformity of up to +/−10% or better. In certain embodiments, the layer of uniform thickness sample has a thickness uniformity of up to +/−20% or better. In certain embodiments, the layer of uniform thickness sample has a thickness uniformity of up to +/−30% or better. In certain embodiments, the layer of uniform thickness sample has a thickness uniformity of up to +/−40% or better. In certain embodiments, the layer of uniform thickness sample has a thickness uniformity of up to +/−50% or better.

In certain embodiments, the spacers are pillars with a cross-sectional shape selected from round, polygonal, circular, square, rectangular, oval, elliptical, or any combination of the same. In certain embodiments, the spacers have pillar shape, have a substantially flat top surface, and have substantially uniform cross-section, wherein, for each spacer, the ratio of the lateral dimension of the spacer to its height is at least 1. In certain embodiments, the inter spacer distance is periodic. In certain embodiments, the spacers have a filling factor of 1% or higher, wherein the filling factor is the ratio of the spacer contact area to the total plate area. In certain embodiments, the Young's modulus of the spacers times the filling factor of the spacers is equal or larger than 20 MPa, wherein the filling factor is the ratio of the spacer contact area to the total plate area. In certain embodiments, the spacing between the two plates at the closed configuration is in less 200 um. In certain embodiments, the spacing between the two plates at the closed configuration is a value selected from between 1.8 um and 3.5 um. In certain embodiments, the spacing are fixed on a plate by directly embossing the plate or injection molding of the plate. In certain embodiments, the materials of the plate and the spacers are selected from polystyrene, PMMA, PC, COC, COP, or another plastic. In certain embodiments, the spacers have a pillar shape, and the sidewall corners of the spacers have a round shape with a radius of curvature at least 1 μm. In certain embodiments, the spacers have a density of at least 1000/mm². In certain embodiments, at least one of the plates is transparent. In certain embodiments, the mold used to make the spacers is fabricated by a mold containing features that are fabricated by either (a) directly reactive ion etching or ion beam etched or (b) by a duplication or multiple duplication of the features that are reactive ion etched or ion beam etched.

In certain embodiments, the spacers are configured, such that the filling factor is in the range of 1% to 5%. In certain embodiments, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%. In certain embodiments, the spacers are configured, such that the filling factor is in the range of 1% to 5%. In certain embodiments, the spacers are configured, such that the filling factor is in the range of 5% to 10%. In certain embodiments, the spacers are configured, such that the filling factor is in the range of 10% to 20%. In certain embodiments, the spacers are configured, such that the filling factor is in the range of 20% to 30%. In certain embodiments, the spacers are configured, such that the filling factor is 5%, 10%, 20%, 30%, 40%, 50%, or in a range of any two of the values. In certain embodiments, the spacers are configured, such that the filling factor is 50%, 60%, 70%, 80%, or in a range of any two of the values.

In certain embodiments, the spacers are configured, such that the filling factor multiplies the Young's modulus of the spacer is in the range of 2 MPa and 10 MPa. In certain embodiments, the spacers are configured, such that the filling factor multiplies the Young's modulus of the spacer is in the range of 10 MPa and 20 MPa. In certain embodiments, the spacers are configured, such that the filling factor multiplies the Young's modulus of the spacer is in the range of 20 MPa and 40 MPa. In certain embodiments, the spacers are configured, such that the filling factor multiplies the Young's modulus of the spacer is in the range of 40 MPa and 80 MPa. In certain embodiments, the spacers are configured, such that the filling factor multiplies the Young's modulus of the spacer is in the range of 80 MPa and 120 MPa. In certain embodiments, the spacers are configured, such that the filling factor multiplies the Young's modulus of the spacer is in the range of 120 MPa to 150 MPa.

In certain embodiments, the device further comprises a dry reagent coated on one or both plates. In certain embodiments, the device further comprises, on one or both plates, a dry binding site that has a predetermined area, wherein the dry binding site binds to and immobilizes an analyte in the sample. In certain embodiments, the device further comprises, on one or both plates, a releasable dry reagent and a release time control material that delays the time that the releasable dry regent is released into the sample. In certain embodiments, the release time control material delays the time that the dry regent starts is released into the sample by at least 3 seconds. In certain embodiments, the regent comprises anticoagulant and/or staining reagent(s). In certain embodiments, the reagent comprises cell lysing reagent(s). In certain embodiments, the device further comprises, on one or both plates, one or a plurality of dry binding sites and/or one or a plurality of reagent sites. In certain embodiments, the analyte comprises a molecule (e.g., a protein, peptides, DNA, RNA, nucleic acid, or other molecule), cells, tissues, viruses, and nanoparticles with different shapes. In certain embodiments, the analyte comprises white blood cells, red blood cells and platelets. In certain embodiments, the analyte is stained.

In certain embodiments, the spacers regulating the layer of uniform thickness have a filling factor of at least 1%, wherein the filling factor is the ratio of the spacer area in contact with the layer of uniform thickness to the total plate area in contact with the layer of uniform thickness. In certain embodiments, for spacers regulating the layer of uniform thickness, the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness. In certain embodiments, for a flexible plate, the thickness of the flexible plate times the Young's modulus of the flexible plate is in the range 60 to 750 GPa-um. In certain embodiments, for a flexible plate, the fourth power of the inter-spacer-distance (ISD) divided by the thickness of the flexible plate (h) and the Young's modulus (E) of the flexible plate, ISD⁴/(hE), is equal to or less than 10⁶ um³/GPa.

In certain embodiments, one or both plates comprises a location marker, either on a surface of or inside the plate, that provide information of a location of the plate. In certain embodiments, one or both plates comprises a scale marker, either on a surface of or inside the plate, that provide information of a lateral dimension of a structure of the sample and/or the plate. In certain embodiments, one or both plates comprises an imaging marker, either on surface of or inside the plate, that assists an imaging of the sample. In certain embodiments, the spacers functions as a location marker, a scale marker, an imaging marker, or any combination of thereof.

In certain embodiments, the average thickness of the layer of uniform thickness is about equal to a minimum dimension of an analyte in the sample. In certain embodiments, the inter-spacer distance is in the range of 7 μm to 50 μm. In certain embodiments, the inter-spacer distance is in the range of 50 μm to 120 μm. In certain embodiments, the inter-spacer distance is in the range of 120 μm to 200 μm (micron). In certain embodiments, the inter-spacer distance is substantially periodic. In certain embodiments, the spacers are pillars with a cross-sectional shape selected from round, polygonal, circular, square, rectangular, oval, elliptical, or any combination of the same.

In certain embodiments, the spacers have a pillar shape and have a substantially flat top surface, wherein, for each spacer, the ratio of the lateral dimension of the spacer to its height is at least 1. In certain embodiments, each spacer has the ratio of the lateral dimension of the spacer to its height is at least 1. In certain embodiments, the minimum lateral dimension of spacer is less than or substantially equal to the minimum dimension of an analyte in the sample. In certain embodiments, the minimum lateral dimension of spacer is in the range of 0.5 um to 100 um. In certain embodiments, the minimum lateral dimension of spacer is in the range of 0.5 um to 10 um.

In certain embodiments, the sample is blood. In certain embodiments, the sample is whole blood without dilution by liquid. In certain embodiments, the sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine. In certain embodiments, the sample is a biological sample, an environmental sample, a chemical sample, or clinical sample.

In certain embodiments, the spacers have a pillar shape, and the sidewall corners of the spacers have a round shape with a radius of curvature at least 1 μm. In certain embodiments, the spacers have a density of at least 100/mm². In certain embodiments, the spacers have a density of at least 1000/mm². In certain embodiments, at least one of the plates is transparent. In certain embodiments, at least one of the plates is made from a flexible polymer. In certain embodiments, for a pressure that compresses the plates, the spacers are not compressible and/or, independently, only one of the plates is flexible. In certain embodiments, the flexible plate has a thickness in the range of 10 um to 200 um. In certain embodiments, the variation is less than 30%. In certain embodiments, the variation is less than 10%. In certain embodiments, the variation is less than 5%.

In certain embodiments, the first and second plates are connected and are configured to be changed from the open configuration to the closed configuration by folding the plates. In certain embodiments, the first and second plates are connected by a hinge and are configured to be changed from the open configuration to the closed configuration by folding the plates along the hinge. In certain embodiments, the first and second plates are connected by a hinge that is a separate material to the plates, and are configured to be changed from the open configuration to the closed configuration by folding the plates along the hinge. In certain embodiments, the first and second plates are made in a single piece of material and are configured to be changed from the open configuration to the closed configuration by folding the plates. In certain embodiments, the layer of uniform thickness sample is uniform over a lateral area that is at least 1 mm².

In certain embodiments, the device is configured to analyze the sample in 60 seconds or less. In certain embodiments, at the closed configuration, the final sample thickness device is configured to analyze the sample in 60 seconds or less. In certain embodiments, at the closed configuration, the final sample thickness device is configured to analyze the sample in 10 seconds or less.

In certain embodiments, the dry binding site comprises a capture agent. In certain embodiments, the dry binding site comprises an antibody or nucleic acid. In certain embodiments, the releasable dry reagent is a labeled reagent. In certain embodiments, the releasable dry reagent is a fluorescently-labeled reagent. In certain embodiments, the releasable dry reagent is a fluorescently-labeled antibody. In certain embodiments, the releasable dry reagent is a cell stain. In certain embodiments, the releasable dry reagent is a cell lysing.

In certain embodiments, the detector is an optical detector that detects an optical signal. In certain embodiments, the detector is an electric detector that detect electrical signal. In certain embodiments, the spacing are fixed on a plate by directly embossing the plate or injection molding of the plate. In certain embodiments, the materials of the plate and the spacers are selected from polystyrene, PMMA, PC, COC, COP, or another plastic.

In certain embodiments of the present disclosure, a system for rapidly analyzing a sample using a mobile phone can comprise a device of any prior embodiment. In certain embodiments of the present disclosure, a system for rapidly analyzing a sample using a mobile phone can comprise a mobile communication device. In certain embodiments, the mobile communication device can comprise one or a plurality of cameras for the detecting and/or imaging the sample. In certain embodiments, the mobile communication device can comprise electronics, signal processors, hardware and software for receiving and/or processing the detected signal and/or the image of the sample and for remote communication. In certain embodiments, the mobile communication device can comprise a light source from either the mobile communication device or an external source. In same embodiments, the detector in the devices or methods of any prior embodiment is provided by the mobile communication device, and detects an analyte in the sample at the closed configuration.

In certain embodiments, one of the plates has a binding site that binds an analyte, wherein at least part of the uniform sample thickness layer is over the binding site, and is substantially less than the average lateral linear dimension of the binding site. In certain embodiments, any system of the present disclosure can comprise a housing configured to hold the sample and to be mounted to the mobile communication device. In certain embodiments, the housing comprises optics for facilitating the imaging and/or signal processing of the sample by the mobile communication device, and a mount configured to hold the optics on the mobile communication device. In certain embodiments, an element of the optics in the housing is movable relative to the housing. In certain embodiments, the mobile communication device is configured to communicate test results to a medical professional, a medical facility or an insurance company. In certain embodiments, the mobile communication device is further configured to communicate information on the test and the subject with the medical professional, medical facility or insurance company. In certain embodiments, the mobile communication device is further configured to communicate information of the test to a cloud network, and the cloud network process the information to refine the test results. In certain embodiments, the mobile communication device is further configured to communicate information of the test and the subject to a cloud network, the cloud network process the information to refine the test results, and the refined test results will send back the subject. In certain embodiments, the mobile communication device is configured to receive a prescription, diagnosis or a recommendation from a medical professional. In certain embodiments, the mobile communication device is configured with hardware and software to capture an image of the sample. In certain embodiments, the mobile communication device is configured with hardware and software to analyze a test location and a control location in in image. In certain embodiments, the mobile communication device is configured with hardware and software to compare a value obtained from analysis of the test location to a threshold value that characterizes the rapid diagnostic test.

In certain embodiments of the present disclosure, at least one of the plates comprises a storage site in which assay reagents are stored. In certain embodiments, at least one of the cameras reads a signal from the device. In certain embodiments, the mobile communication device communicates with the remote location via a wifi or cellular network. In certain embodiments, the mobile communication device is a mobile phone.

In certain embodiments of the present disclosure, a method for rapidly analyzing an analyte in a sample using a mobile phone can comprise depositing a sample on the device of any prior system embodiment. In certain embodiments of the present disclosure, a method for rapidly analyzing an analyte in a sample using a mobile phone can comprise assaying an analyte in the sample deposited on the device to generate a result. In certain embodiments of the present disclosure, a method for rapidly analyzing an analyte in a sample using a mobile phone can comprise communicating the result from the mobile communication device to a location remote from the mobile communication device.

In certain embodiments, the analyte comprises a molecule (e.g., a protein, peptides, DNA, RNA, nucleic acid, or other molecule), cells, tissues, viruses, and nanoparticles with different shapes. In certain embodiments, the analyte comprises white blood cell, red blood cell and platelets. In certain embodiments, the assaying comprises performing a white blood cells differential assay. In certain embodiments, a method of the present disclosure can comprise analyzing the results at the remote location to provide an analyzed result. In certain embodiments, a method of the present disclosure can comprise communicating the analyzed result from the remote location to the mobile communication device. In certain embodiments, the analysis is done by a medical professional at a remote location. In certain embodiments, the mobile communication device receives a prescription, diagnosis or a recommendation from a medical professional at a remote location.

In certain embodiments, the sample is a bodily fluid. In certain embodiments, the bodily fluid is blood, saliva or urine. In certain embodiments, the sample is whole blood without dilution by a liquid. In certain embodiments, the assaying step comprises detecting an analyte in the sample. In certain embodiments, the analyte is a biomarker. In certain embodiments, the analyte is a protein, nucleic acid, cell, or metabolite. In certain embodiments, the method comprises counting the number of red blood cells. In certain embodiments, the method comprises counting the number of white blood cells. In certain embodiments, the method comprises staining the cells in the sample and counting the number of neutrophils, lymphocytes, monocytes, eosinophils and basophils. In certain embodiments, the assay done in step (b) is a binding assay or a biochemical assay.

In certain embodiments of the present disclosure, a method for analyzing a sample can comprise obtaining a device of any prior device embodiment. In certain embodiments of the present disclosure, a method for analyzing a sample can comprise depositing the sample onto one or both pates of the device. In certain embodiments of the present disclosure, a method for analyzing a sample can comprise placing the plates in a closed configuration and applying an external force over at least part of the plates. In certain embodiments of the present disclosure, a method for analyzing a sample can comprise analyzing the layer of uniform thickness while the plates are the closed configuration.

In certain embodiments, the first plate further comprises, on its surface, a first predetermined assay site and a second predetermined assay site, wherein the distance between the edges of the assay site is substantially larger than the thickness of the uniform thickness layer when the plates are in the closed position, wherein at least a part of the uniform thickness layer is over the predetermined assay sites, and wherein the sample has one or a plurality of analytes that are capable of diffusing in the sample. In certain embodiments, the first plate has, on its surface, at least three analyte assay sites, and the distance between the edges of any two neighboring assay sites is substantially larger than the thickness of the uniform thickness layer when the plates are in the closed position, wherein at least a part of the uniform thickness layer is over the assay sites, and wherein the sample has one or a plurality of analytes that are capable of diffusing in the sample. In certain embodiments, the first plate has, on its surface, at least two neighboring analyte assay sites that are not separated by a distance that is substantially larger than the thickness of the uniform thickness layer when the plates are in the closed position, wherein at least a part of the uniform thickness layer is over the assay sites, and wherein the sample has one or a plurality of analytes that are capable of diffusing in the sample. In certain embodiments, the analyte assay area is between a pair of electrodes. In certain embodiments, the assay area is defined by a patch of dried reagent. In certain embodiments, the assay area binds to and immobilizes the analyte. In certain embodiments, the assay area is defined by a patch of binding reagent that, upon contacting the sample, dissolves into the sample, diffuses in the sample, and binds to the analyte. In certain embodiments, the inter-spacer distance is in the range of 14 μm to 200 μm. In certain embodiments, the inter-spacer distance is in the range of 7 μm to 20 μm. In certain embodiments, the spacers are pillars with a cross-sectional shape selected from round, polygonal, circular, square, rectangular, oval, elliptical, or any combination of the same. In certain embodiments, the spacers have a pillar shape and have a substantially flat top surface, wherein, for each spacer, the ratio of the lateral dimension of the spacer to its height is at least 1. In certain embodiments, the spacers have a pillar shape, and the sidewall corners of the spacers have a round shape with a radius of curvature at least 1 μm. In certain embodiments, the spacers have a density of at least 1000/mm². In certain embodiments, at least one of the plates is transparent. In certain embodiments, at least one of the plates is made from a flexible polymer. In certain embodiments, only one of the plates is flexible. In certain embodiments, the area-determination device is a camera. In certain embodiments, an area in the sample contact area of a plate, wherein the area is less than 1/100, 1/20, 1/10, ⅙, ⅕, ¼, ⅓, ½, ⅔ of the sample contact area, or in a range between any of the two values. In certain embodiments, the area-determination device comprises a camera and an area in the sample contact area of a plate, wherein the area is in contact with the sample.

In certain embodiments, the deformable sample comprises a liquid sample. In certain embodiments, the imprecision force has a variation at least 30% of the total force that actually is applied. In certain embodiments, the imprecision force has a variation at least 20%, 30%, 40%, 50%, 60, 70%, 80%, 90% 100%, 150%, 200%, 300%, 500%, or in a range of any two values, of the total force that actually is applied. In certain embodiments, the spacers have a flat top. In certain embodiments, the device is further configured to have, after the pressing force is removed, a sample thickness that is substantially the same in thickness and uniformity as that when the force is applied. In certain embodiments, the imprecise force is provided by human hand. In certain embodiments, the inter spacer distance is substantially constant. In certain embodiments, the inter spacer distance is substantially periodic in the area of the uniform sample thickness area. In certain embodiments, the multiplication product of the filling factor and the Young's modulus of the spacer is 2 MPa or larger. In certain embodiments, the force is applied by hand directly or indirectly. In certain embodiments, the force applied is in the range of 1 N to 20 N. In certain embodiments, the force applied is in the range of 20 N to 200 N. In certain embodiments, the highly uniform layer has a thickness that varies by less than 15%, 10%, or 5% of an average thickness. In certain embodiments, the imprecise force is applied by pinching the device between a thumb and forefinger. In certain embodiments, the predetermined sample thickness is larger than the spacer height. In certain embodiments, the device holds itself in the closed configuration after the pressing force has been removed. In certain embodiments, the uniform thickness sample layer area is larger than that area upon which the pressing force is applied. In certain embodiments, the spacers do not significantly deform during application of the pressing force. In certain embodiments, the pressing force is not predetermined beforehand and is not measured. In certain embodiments, the fluidic sample is replaced by a deformable sample and the embodiments for making at least a part of the fluidic sample into a uniform thickness layer can make at least a part of the deformable sample into a uniform thickness layer. In certain embodiments, the inter spacer distance is periodic. In certain embodiments, the spacers have a flat top. In certain embodiments, the inter spacer distance is at least two times large than the size of the targeted analyte in the sample.

### Manufacturing of Q-Card

In certain embodiments of the present disclosure, a Q-Card can comprise a first plate. In certain embodiments of the present disclosure, a Q-Card can comprise a second plate. In certain embodiments of the present disclosure, a Q-Card can comprise a hinge. In certain embodiments, the first plate, that is about 200 nm to 1500 nm thick, comprises, on its inner surface, (a) a sample contact area for contacting a sample, and (b) a sample overflow dam that surrounds the sample contact area is configured to present a sample flow outside of the dam. In certain embodiments, the second plate is 10 um to 250 um thick and comprises, on its inner surface, (a) a sample contact area for contacting a sample, and (b) spacers on the sample contact area. In certain embodiments, the hinge that connect the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, (a) a sample contact area for contacting a sample, (b) a sample overflow dam that surrounds the sample contact area is configured to present a sample flow outside of the dam, and (c) spacers on the sample contact area. In certain embodiments, the second plate, that is 10 um to 250 um thick, comprises, on its inner surface, a sample contact area for contacting a sample. In certain embodiments, the hinge connects the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, (a) a sample contact area for contacting a sample, and (b) spacers on the sample contact area. In certain embodiments, the second plate, that is 10 um to 250 um thick, comprises, on its inner surface, (a) a sample contact area for contacting a sample, and (b) a sample overflow dam that surrounds the sample contact area is configured to present a sample flow outside of the dam. In certain embodiments, the hinge connects the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, a sample contact area for contacting a sample. In certain embodiments, the second plate, that is 10 um to 250 um thick, comprises, on its inner surface, (a) a sample contact area for contacting a sample, (b) a sample overflow dam that surrounds the sample contact area is configured to present a sample flow outside of the dam, and (c) spacers on the sample contact area. In certain embodiments, the hinge connects the first and the second plates. In certain embodiments, the first and second plate are movable relative to each other around the axis of the hinge.

In certain embodiments of the present disclosure, a method for fabricating any Q-Card of the present disclosure can comprise injection molding of the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise Laser cutting the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise injection molding and laser cutting the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise nanoimprinting or extrusion printing to fabricated both the first and the second plate.

In certain embodiments of the present disclosure, a method for fabricating any Q-Card of the present disclosure can comprise fabricating the first plate or the second plate, using injection molding, laser cutting the first plate, nanoimprinting, extrusion printing, or a combination of thereof.

In certain embodiments of the present disclosure, a method for fabricating any Q-Card of the present disclosure can comprise a step of attaching the hinge on the first and the second plates after the fabrication of the first and second plates.

### 6. EXAMPLES OF THE PRESENT INVENTION

### Example 1 Preparation of QMAX Device

Using the manufacture protocols of Q-cards described above, we prepared a QMAX device. The QMAX device includes two plates. A first plate was made with a dimension of 24 mm×32 mm×1 mm in size. A second plate was made of a dimension of 22 mm×27 mm×175 mm in size. On the first plate, a plurality of spacers was created to have the dimension of 30 μm×40 μm×5 μm in size, and each spacer is separated by an 80 μm inter-spacer distance.

### Example 2 QMAX Device Includes a Fluorescent Label for Counting Platelets in a Blood Sample (without Lysing Red Blood Cells)

In this study, we used the QMAX device as prepared in Example 1. Using a droplet printing process, a 15 mm×15 mm array of 11 nL sized droplets of fluorescent dye (fluorescent label) was printed with a 0.65 mm period onto the second plate. Each droplet printed onto the second plate included a 0.4 mg/mL concentration of the Acridine orange dye. We also printed onto the second plate with a 0.15 mg/mL concentration of Zwittergent. Note that at this concentration, we observed that the Zwittergent at that concentration did not cause lysis of red blood cells, but provided a better result of imaging.

Human whole blood (5 μL) was obtained and immediately deposited onto the second plate. The first plate and the second plate were gently pressed together to allow the blood sample came into contact with the coated fluorescent dye (on the first plate) within 5 min. Without washing, images of the fluorescent staining were taken using iPhone. The fluorescent staining was analyzed which is an indicative of the platelet counts.

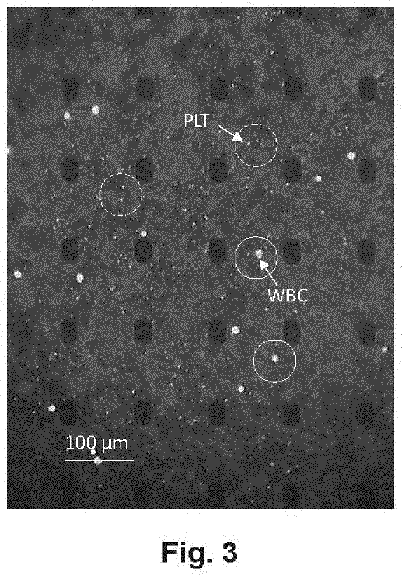

As shown in FIG. 3, the platelets were readily visible and were smaller as compared to that that of white blood cells (circled). The platelet count was analyzed. Note that the red blood cells can be seen in the background and they were intact.

### Example 3 QMAX Device Including a Fluorescent Label for Counting Platelets in a Blood Sample (with Lysing Red Blood Cells)

In this study, we used the QMAX device as prepared in Example 1. Using a droplet printing process, a 15 mm×15 mm array of 11 nL sized droplets was printed with a 0.65 mm period onto the second plate. Each droplet printed onto the second plate included a concentration of YOYO in the range of 5-20 μM and a concentration of Zwittergent in the range of 0.5-2.0 mg/mL. Note that at this concentration range, the Zwittergent could readily cause the red blood cell lysis when it contacted with the blood sample. It is found that lysis of red blood cells provided a clearer background for better fluorescent images.

Here, human whole blood (5 μL) was obtained and immediately deposited onto the second plate. The first plate and the second plate were gently pressed together to allow the blood sample came into contact with the coated fluorescent dye (on the first plate) within 5 min. Without washing, images of the fluorescent staining were taken using iPhone. The fluorescent staining was analyzed which is an indicative of the platelet counts.

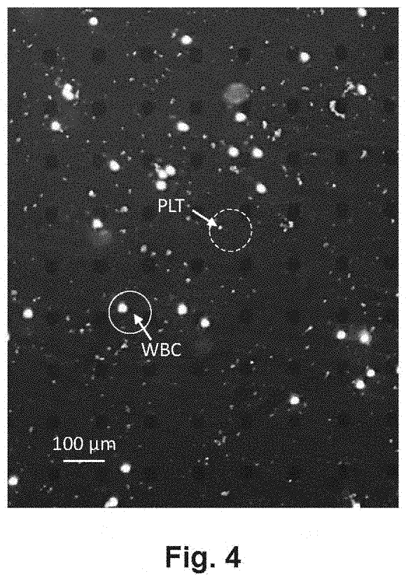

As shown in FIG. 4, the platelet were readily viewed, together with the white blood cells (circled). The image was analyzed for platelet counts. Note that there were no red blood cells in the background (as compared to Example 3 above) because they were lysed with Zwittergent (0.5-2.0 mg/mL).

### Example 4 QMAX Device Including a Calorimetric Label for Counting Platelets in a Blood Sample (with Lysing Red Blood Cells)

We used the QMAX device as prepared in Example 1. Using a droplet printing process, a 15 mm×15 mm array of 11 nL sized droplets was printed with a 0.65 mm period onto the second plate.

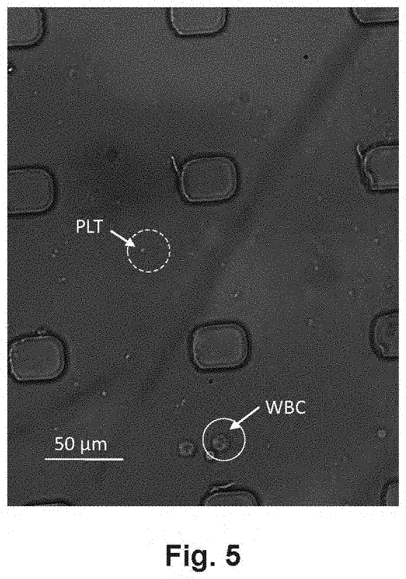

In this study, we used a calorimetric dye (calorimetric label) of methylene blue to stain platelets (instead of a fluorescent dye). Each droplet printed onto the second plate included a concentration of methylene blue dye in the range of 0.01-0.05% and a concentration of Zwittergent in the range of 0.5-2.0 mg/mL. As shown in FIG. 5, there were little platelet staining, indicating the majority of the platelets in the sample was alive. The methylene blue is reduced in viable cells (platelets), leaving them unstained. However dead platelets are unable to reduce the oxidized methylene blue and the platelets are stained blue.

Note that at this concentration range, the Zwittergent could readily cause the red blood cell lysis when it contacted with the blood sample. It is found that lysis of red blood cells provided a clearer background for better calorimetric images (See, FIG. 5).

### Example 5 Decay of Platelet Staining Over Time

In this series of study, we examined the effect of aged blood on platelet staining.

We used the QMAX device as prepared in Example 1. Using a droplet printing process, a 15 mm×15 mm array of 11 nL sized droplets of fluorescent dye (fluorescent label) was printed with a 0.65 mm period onto the second plate. Each droplet printed onto the second plate included a 0.4 mg/mL concentration of the Acridine orange dye. We also printed onto the second plate with a 0.2-2 mg/mL concentration of Zwittergent.

Human whole blood (5 μL) was obtained and deposited onto the second plate at different time points. After deposition, the first plate and the second plate were gently pressed together to allow the blood sample came into contact with the coated fluorescent dye (on the first plate). Without washing, images of the fluorescent staining were taken using iPhone. The fluorescent staining was analyzed which is an indicative of the platelet counts.

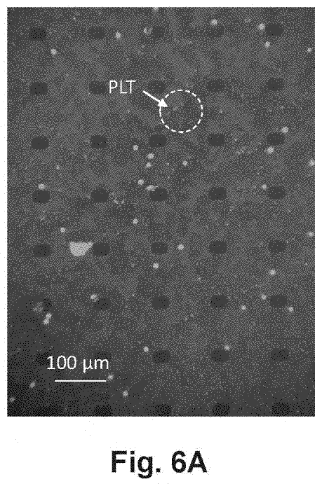

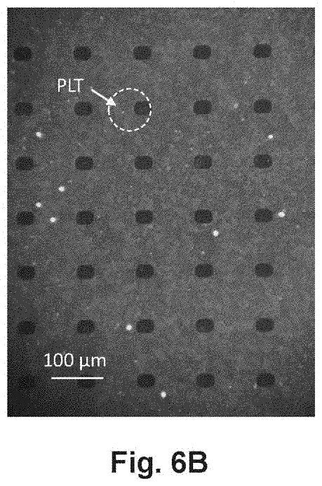

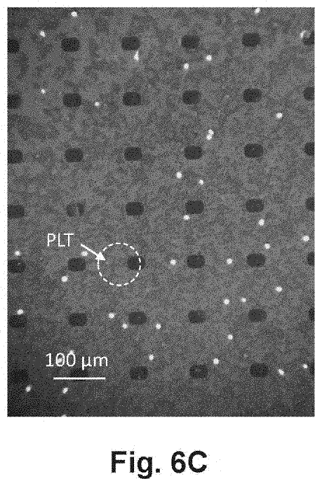

As shown in FIGS. 6A, 6B, and 6C, images of the results of platelet test using device having fluorescence labels and no RBC lysing agent were obtained after 1 minute, 30 minutes, and 180 minutes between the blood sample was drawn. The time interval was defined between the time when the blood sample was obtained and allowed to come into contact with the staining reagent by pressing the first plate and the second plate together after blood deposition.

Of interest is our observation that the fluorescent staining (e.g., Acridine Orange dye) of the DNA/RNA present in the platelets decreased with time. A rapid (occurred after 10 minutes) and significant decrease (˜50% decrease) was observed. This study indicates that for accurate platelet counting using fluorescent staining, it is essential to use freshly isolated blood (i.e., not aged blood). Preferably, the blood exceeded over 20 min should be discarded. On the other hand, one advantage of the present device is to evaluate if the blood sample has been aged (e.g., sitting at room temperature for a while (e.g., over 10 min), after withdrawn from a human).

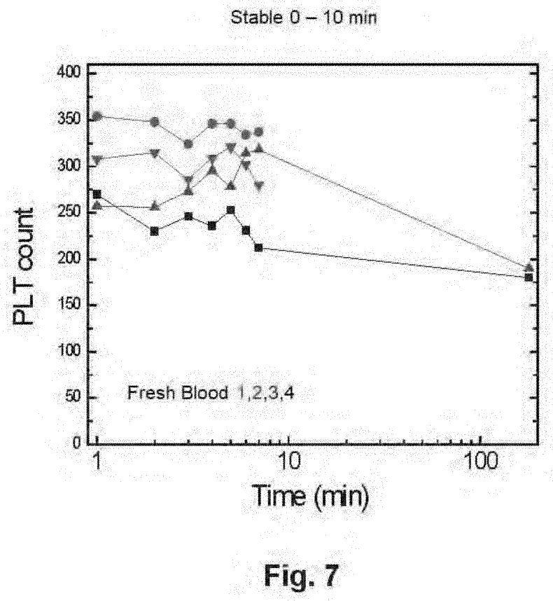

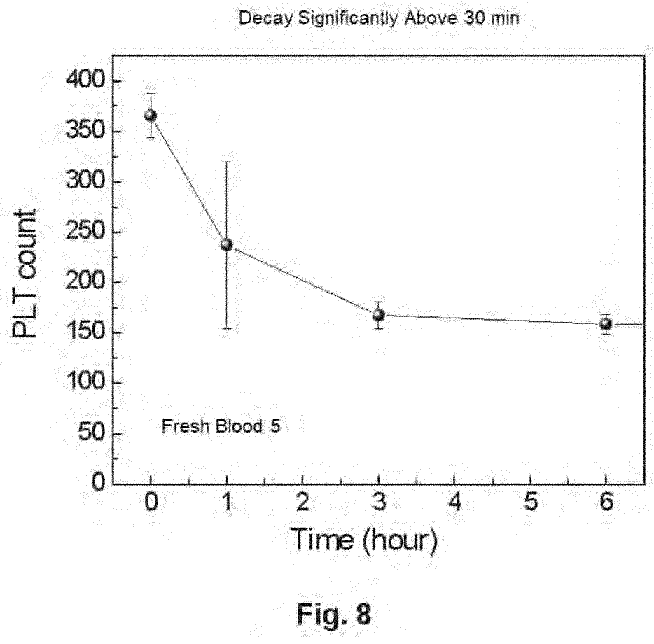

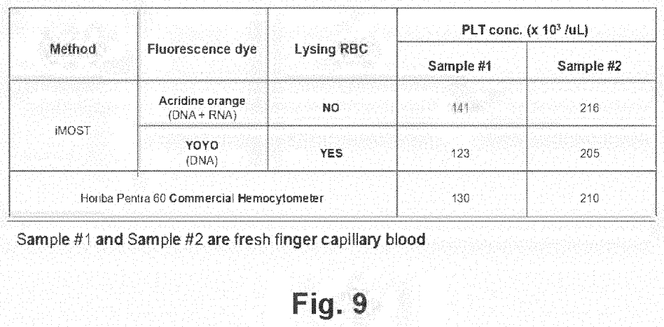

The kinetics of the decay in platelet staining using acridine orange was evaluated. As shown in FIG. 7, the decay of the platelet staining agent was found to be stable over 10 minutes. As shown in FIG. 8, decay of the platelet staining agent was noted to be significant above 30 minutes. As shown in the Table in FIG. 9, we compared the platelet counting method using the device having a fluorescence label and no RBC lysing, the platelet counting method using the device having a fluorescen label with RBC lysing, and the platelet counting method using a commercial hemocytometer.

What is claimed is:

1. A device for assessing platelet quality in a blood sample, comprising:
a first plate; a second plate; spacers; and a viability dye, wherein:
i. the plates are movable relative to each other into different configurations, including an open configuration and a closed configuration,
ii. each of the plates has, on its respective sample surface, a sample contact area for contacting a blood sample that includes platelets,
iii. one or both of the plates comprises the spacers, and the spacers are fixed to the respective sample contact area,
iv. each of the spacers has a pillar shape, a flat top, a predetermined substantially uniform height in the range of 0.5 μm to 6 μm, and a predetermined constant inter-spacer distance, and
v. the viability dye is coated on one or both of the plates, on their respective sample surfaces, and configured to stain the non-viable platelets of the blood sample and generate an optical signal indicative of a viability state of each of the stained platelets upon exposure to predetermined first wavelengths of light,
wherein in the open configuration, the two plates are partially or entirely separated apart, the spacing between the plates is not regulated by the spacers, and the blood sample is deposited on one or both of the plates, and
wherein in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers.

2. A kit for assessing platelet quality in a blood sample, comprising:
a first plate; a second plate; spacers; and a viability dye, wherein:
a) the plates are movable relative to each other into different configurations, including an open configuration and a closed configuration,
b) each of the plates has, on its respective sample surface, a sample contact area for contacting a blood sample that includes platelets,
c) one or both of the plates comprises the spacers, and the spacers are fixed to the respective sample contact area,
d) the spacers have a predetermined substantially uniform height in the range of 0.5 μm to 6 μm, and a predetermined constant inter-spacer distance, and
e) the viability dye is configured to stain the non-viable platelets of the blood sample and generate an optical signal indicative of a viability state of each of the stained platelets upon exposure to predetermined first wavelengths of light,
wherein in the open configuration, the two plates are partially or entirely separated apart, the spacing between the plates is not regulated by the spacers, and the blood sample is deposited on one or both of the plates, and
wherein in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers.

3. A system for assessing platelet quality in a blood sample, comprising:
a) the device of claim 1;
b) an imager, comprising a camera and a light source for imaging the platelets in the layer of uniform thickness upon exposure to wavelengths of light that include at least the predetermined first wavelengths; and
c) a processor, comprising electronics, signal processors, hardware and software for receiving and processing the images and identifying and analyzing the platelets in the images.

4. A method of assessing platelet quality in a blood sample, comprising the steps of:
a) obtaining a blood sample that includes platelets;
b) obtaining a first plate and a second plate that are movable relative to each other into different configurations including an open configuration and a closed configuration, wherein:
i) each of the plates has a sample contact area on its respective sample surface for contacting the blood sample, and
ii) one or both of the plates comprises spacers that are fixed to its sample contact area,
wherein the spacers have a predetermined substantially uniform height in the range of 0.5 μm to 2.5 μm, and a predetermined constant inter-spacer distance,
c) depositing the sample on one or both of the plates when the plates are in an open configuration, wherein in the open configuration the two plates are partially or entirely separated apart and the spacing between the plates is not regulated by the spacers;
d) before or after (c), mixing the blood sample with a viability dye that is configured to stain the non-viable platelets and generate an optical signal indicative of a viability state of each of the stained platelets upon exposure to predetermined first wavelengths of light;
e) after (c) and (d), bringing the two plates together and pressing the plates into a closed configuration, wherein in the closed configuration at least part of the blood sample is compressed by the two plates into a layer of highly uniform thickness and the uniform thickness of the layer is confined by the sample contact areas of the two plates and is regulated by the spacers and the plates;
f) while the plates are at the closed configuration, acquiring a bright-field image of the platelets and an image of the optical signals of the platelets rendered by the viability dye in the layer of uniform thickness; and
g) identifying the platelets in the acquired images and assessing the quality of the platelets.

5. The device, kit, system, or method of any prior claim, wherein the viability dye selected from the group consisting of Propidium Iodide, 7-AAD, Trypan blue, Calcein Violet AM, Calcein AM, Fixable Viability Dyes, SYTO9 and other nucleic acid dyes, Resazurin and Formazan (MTT/XTT), other mitochondrial dyes, and any combination thereof.

6. The device, kit, system, or method of any prior claim, wherein the viability dye is coated on the sample contact area of one or both of the plates and configured to, upon contacting the blood sample, be dissolved and diffuse in the blood sample.

7. The device, kit, system, or method of any prior claim, wherein the viability dye is fluorescently labeled and the optical signal is a fluorescent signal.

8. The device, kit, system, or method of any prior claim, wherein the viability dye is a colorant and renders the stained platelets of different viability states different colors.

9. The device, kit, system, or method of any prior claim, wherein the optical signal is:
i) fluorescence;
ii) light absorption, reflection, transmission, diffraction, scattering, or diffusion;
iii) surface Raman scattering; or
iv) any combination of i-iii.

10. The device, kit, system, or method of any prior claim, wherein the height of the spacers is around 2 μm.

11. The device, kit, system, or method of any prior claim, wherein the height of the spacers is selected such that in the closed configuration, a substantial fraction of RBCs in the layer of uniform thickness are lysed, and a substantial fraction of the platelets in the layer of uniform thickness are not lysed.

12. The device, kit, system, or method of any prior claim, wherein the height of the spacers is in the range of 0.5 μm to 1.2 μm.

13. The device, kit, system, or method of any prior claim, wherein on one or both the sample contact areas, the respective plate further comprises a layer of a lysing agent that facilitates the lysing of RBCs, WBCs, or other cells in the blood sample.

14. The device, kit, system, or method of any prior claim, wherein one or both the sample contact areas of the respective plate further comprises a layer of a reagent for bio/chemical assay of the platelets.

15. The device, kit, system, or method of any prior claim, wherein the lysing agent is selected from the group consisting of ammonium chloride, organic quaternary ammonium surfactants, cyanide salts, and any combination thereof.

16. The device, kit, system, or method of any prior claim, wherein the substantial fraction is at least 51%, 60%, 70%, 80%, 90%, 95% or 99% of a component in the relevant volume of the sample.

17. The device, kit, system, or method of any prior claim, wherein the thickness variation of the layer of highly uniform thickness over the lateral area of the relevant volume is equal to or less than 40%, 30%, 20%, 15%, 10%, 7%, 5%, 3%, or 1%, or in a range between any of the two values, wherein the thickness variation is relative to the average thickness of the lateral area.

18. The device, kit, system, or method of any prior claim, wherein the area of the highly uniform layer is equal to or larger than 0.1 mm², 0.5 mm², 1 mm², 3 mm², 5 mm², 10 mm², 20 mm², 50 mm², 70 mm², 100 mm², 200 mm², 500 mm², 800 mm², 1000 mm², 2000 mm², 5000 mm², 10000 mm², 20000 mm², 50000 mm², or 100000 mm²; or in a range between any of the two values.

19. The device, kit, system, or method of any prior claim, wherein the blood sample is diluted or undiluted whole blood.

20. The device, kit, system, or method of any prior claim, wherein the blood sample is a partial blood sample.

21. The device, kit, system, or method of any prior claim, wherein the spacer height is equal to or less than 2 μm, 1.9 μm, 1.8 μm, 1.7 μm, 1.6 μm, 1.5 μm, 1.4 μm, 1.3 μm, 1.2 μm, 1.1 μm, 1.0 μm, 0.9 μm, 0.8 μm, 0.7 μm, 0.6 μm, 0.5 μm, 0.4 μm, 0.3 μm, or 0.2 μm, or in a range between any of the two values.

22. The device, kit, system, or method of any prior claim, wherein in the closed configuration, a substantial fraction of white blood cells (WBCs) in the relevant volume of the sample are lysed, and the spacer height is equal to or less than 1.0 μm, 0.9 μm, 0.8 μm, 0.7 μm, 0.6 μm, 0.5 μm, 0.4 μm, 0.3 μm, or 0.2 μm, or in a range between any of the two values.

23. The device, kit, system, or method of any prior claim, wherein at least one of the plates is transparent.

24. The system of any prior claim, wherein the camera and the processor are part of a mobile communication device.

25. The system of claim 24, wherein the light source is an internal light source of the mobile communication device.

26. The system of claim 24, wherein the light source is a light source external to the mobile communication device.

27. The system of any prior claim, wherein the mobile communication device is a mobile phone.

28. The system of any prior claim, further comprising a housing configured to hold the sample and to be mounted to the mobile communication device.

29. The system of any prior claim, wherein the housing comprises optics for facilitating the imaging and/or signal processing of the sample by the mobile communication device and a mount configured to hold the optics on the mobile communication device.

30. The system of any of any prior claim, wherein the mobile communication device is configured to communicate test results to a medical professional, a medical facility or an insurance company.

31. The system of any prior claim, wherein the mobile communication device is further configured to communicate information on the subject with the medical professional, medical facility or insurance company.

32. The system of any prior claim, wherein the mobile communication device is configured to receive a prescription, diagnosis or a recommendation from a medical professional.

33. The system of any prior claim, wherein the mobile communication device communicates with the remote location via a Wi-Fi or cellular network.

34. The system of any prior claim, wherein the mobile communication device is a mobile phone.

35. The method of any prior claim, wherein step (f) comprises the steps of:
i) imaging the platelets in the layer of uniform thickness under bright-field illumination; and
ii) imaging fluorescent signals of the platelets in the layer of uniform thickness rendered by the viability dye upon exposure to the first wavelengths of light.

36. The method of any prior claim, wherein step (g) is performed by:
(i) identifying and obtaining a total number of the platelets in a first area of the acquired images;
(ii) classifying the platelets in the first area, based on the optical signals from individual platelets rendered by the viability dye; and
(iii) quantifying the quality of the platelets by calculating the percentage of the platelets in each class over the total number.

37. The method of claim 36, wherein the step (i) is performed by processing and analyzing the acquired images of the platelets under bright-field illumination to identify and obtain a total number of the platelets in the first area of the bright filed images.

38. The method of any prior claim, wherein the identifying step in step (g) comprises processing and analyzing the images using algorithms for edge detection and circle detection.

39. The method of any prior claim, wherein step (g) is performed by a mobile communication device that is configured to either receive or process the image of the platelets, or both.

40. The method of any prior claim, wherein the viability dye is supplied separately and added into the blood sample before the blood sample is deposited on the plates.

41. The method of any prior claim, wherein the viability dye is coated on the sample contact area of one or both of the plates, and configured to, upon contacting the sample, be dissolved and diffuse in the sample.

42. The device, kit, system, or method of any prior claim, wherein the spacers have:
i. a shape of pillar with substantially uniform cross-section and a flat top surface;
ii. a ratio of the width to the height equal or larger than one;
iii. a filling factor of equal to 1% or larger; and
iv. a product of the filling factor and the Young's modulus of the spacer is 2 MPa or larger,
wherein the filling factor is the ratio of the spacer contact area to the total plate area.

43. The device, kit, system, or method of any prior claim, wherein the spacer height is in the range of 0.5 μm to 62.5 μm.

44. The device, kit, system, or method of any prior claim, wherein a portion of the spacers have a periodic spacing.

45. The device, kit, system, or method of any prior claim, wherein an average value of the uniform thickness of the layer is substantially the same as the uniform height of the spacer with a variation of less than 10%.

46. The device, kit, system, or method of any prior claim, wherein in the closed configuration at least 90% of the RBCs are lysed and at least 90% of the platelets are not lysed.

47. The device, kit, system, or method of any prior claim, wherein in the closed configuration at least 99% of the RBCs are lysed and at least 99% of the platelets are not lysed.

48. The device, kit, system, or method of any prior claim, wherein the variation of the layer of uniform thickness is less than 30 nm.

49. The device, kit, system, or method of any prior claim, wherein the spacers are pillars with a cross-sectional shape selected from the group consisting of round, polygonal, circular, square, rectangular, oval, elliptical, and any combination of the same.

50. The device, kit, system, or method of any prior claim, wherein the spacers have:
i) a shape of pillar with substantially uniform cross-section and a flat top surface;
ii) a ratio of the width to the height equal or larger than one;
iii) a predetermined constant inter-spacer distance that is in the range of 10 μm to 200 μm;
iv) a filling factor of equal to 1% or larger; and
v) a product of the filling factor and the Young's modulus of the spacer is 2 MPa or larger.
wherein the filling factor is the ratio of the spacer contact area to a total plate area.

51. The device, kit, system, or method of any prior claim, wherein pressing the plates into the closed configuration is conducted either in parallel or sequentially, the parallel pressing applies an external force on an intended area at the same time, and the sequential pressing applies an external force on a part of an intended area and gradually moves to another area.

52. The device, kit, system, or method of any prior claim, wherein the blood sample is analyzed by:
i) illuminating at least part of the blood sample in the layer of uniform thickness;
ii) obtaining one or more images of the cells using a CCD or CMOS sensor;
iii) identifying the platelets in the image using a computer; and
iv) counting a number of platelets in an area of the image.

53. The device, kit, system, or method of any prior claim, wherein the layer of uniform thickness has a thickness uniformity of up to +/−5%.

54. A device for quantifying platelets in a blood sample, comprising:
a first plate; a second plate; spacers; and a staining dye, wherein:
a) the plates are movable relative to each other into different configurations, including an open configuration and a closed configuration,
b) each of the plates has, on its respective sample surface, a sample contact area for contacting a blood sample that includes platelets,
c) one or both of the plates comprises the spacers, and the spacers are fixed to the respective sample contact area,
d) the spacers have a predetermined substantially uniform height in the range of 0.2 μm to 6 μm, and a predetermined constant inter-spacer distance, and
e) the staining dye is coated on one or both of the plates, on their respective sample surfaces, and configured to stain the platelets of the blood sample and generate an optical signal indicative of the total amount of platelets in the blood sample upon exposure to predetermined first wavelengths of light,
wherein in the open configuration, the two plates are partially or entirely separated apart, the spacing between the plates is not regulated by the spacers, and the blood sample is deposited on one or both of the plates, and
wherein in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers.

55. A kit for quantifying platelets in a blood sample, comprising:
a first plate; a second plate; spacers; and a staining dye, wherein:
a) the plates are movable relative to each other into different configurations, including an open configuration and a closed configuration,
b) each of the plates has, on its respective sample surface, a sample contact area for contacting a blood sample that includes platelets,
c) one or both of the plates comprises the spacers, and the spacers are fixed to the respective sample contact area,
d) the spacers have a predetermined substantially uniform height in the range of 0.2 μm to 6 μm, and a predetermined constant inter-spacer distance, and
e) the staining dye is configured to stain the platelets of the blood sample and generate an optical signal indicative of the total amount of platelets in the blood sample upon exposure to predetermined first wavelengths of light,
wherein in the open configuration, the two plates are partially or entirely separated apart, the spacing between the plates is not regulated by the spacers, and the blood sample is deposited on one or both of the plates, and
wherein in the closed configuration, which is configured after deposition of the blood sample in the open configuration, at least part of the sample is compressed by the two plates into a layer of highly uniform thickness and the uniform thickness of the layer is confined by the sample surfaces of the plates and is regulated by the plates and the spacers.

56. A system for quantifying platelets in a blood sample, comprising:
a) the device of claim 52 or claim 53;
b) an imager, comprising a camera and a light source for imaging the platelets in the layer of uniform thickness upon exposure to wavelengths of light that include at least the predetermined first wavelengths; and
c) a processor, comprising electronics, signal processors, hardware and software for receiving and processing the images and identifying and analyzing the platelets in the images.

57. A method of quantifying platelets in a blood sample, comprising the steps of:
a) obtaining a blood sample that includes platelets;
b) obtaining a first plate and a second plate that are movable relative to each other into different configurations including an open configuration and a closed configuration, wherein:
iii) each of the plates has a sample contact area on its respective sample surface for contacting the blood sample, and
iv) one or both of the plates comprises spacers that are fixed to its sample contact area,
wherein the spacers have a predetermined substantially uniform height in the range of 0.2 μm to 6 μm, and a predetermined constant inter-spacer distance,
c) depositing the sample on one or both of the plates when the plates are in an open configuration, wherein in the open configuration the two plates are partially or entirely separated apart and the spacing between the plates is not regulated by the spacers;
d) before or after (c), mixing the blood sample with a staining dye that is configured to stain the platelets and generate an optical signal indicative of the total amount of platelets in the blood sample upon exposure to predetermined first wavelengths of light;
e) after (c) and (d), bringing the two plates together and pressing the plates into a closed configuration, wherein in the closed configuration at least part of the blood sample is compressed by the two plates into a layer of highly uniform thickness and the uniform thickness of the layer is confined by the sample contact areas of the two plates and is regulated by the spacers and the plates;
f) while the plates are at the closed configuration, acquiring a bright-field image of the platelets and an image of the optical signals of the platelets rendered by the platelet staining dye in the layer of uniform thickness; and
g) identifying the platelets in the acquired images and assessing the quality of the platelets.

58. The device, kit, system, or method of any prior claim, wherein the spacers have a predetermined substantially uniform height in the range of 2 μm to 5 μm.

59. The device, kit, system, or method of claim 56, wherein the spacers have a predetermined substantially uniform height of 5 μm.

60. The device, kit, system, or method of any prior claim, wherein the staining dye is a dye selected from the group consisting of Acridine orange, YOYO-1, and methylene blue.

61. The device, kit, system, or method of claim 58, wherein the staining dye is Acridine orange.

62. The device, kit, system, or method claim 59, wherein the Acridine orange has a concentration of 0.4 mg/mL.

63. The device, kit, system, or method of claim 58, wherein the staining dye is YOYO-1.

64. The device, kit, system, or method of claim 61, wherein the YOYO-1 has a concentration of 5 μM-20 μM.

65. The device, kit, system, or method of claim 62, wherein the YOYO-1 has a concentration of 10 μM.

66. The device, kit, system, or method of claim 58, wherein the staining dye is methylene blue.

67. The device, kit, system, or method of claim 64, wherein the methylene blue has a concentration of 0.01%-0.05%.

68. The device, kit, system, or method of any prior claim, wherein the staining dye is coated in a 15 mm×15 mm array of droplets having a 0.65 mm period, each droplet having a 11 nL volume.

69. The device, kit, system, or method of claim 66, wherein each droplet contains Acridine orange and 3-[hexadecyl(dimethyl)azaniumyl]propane-1-sulfonate.

70. The device, kit, system, or method of claim 67, wherein each droplet contains a Acridine orange concentration of 0.4 mg/mL and a 3-[hexadecyl(dimethyl) azaniumyl]propane-1-sulfonate concentration of 0.15 mg/m L.

71. The device, kit, system, or method of claim 66, wherein each droplet contains YOYO-1 and 3-[hexadecyl(dimethyl)azaniumyl]propane-1-sulfonate.

72. The device, kit, system, or method of claim 69, wherein each droplet contains a YOYO-1 concentration of 5 μM-20 μM and a 3-[hexadecyl(dimethyl) azaniumyl]propane-1-sulfonate concentration of 0.5 mg/mL-2.0 mg/mL.

73. The device, kit, system, or method of claim 66, wherein each droplet contains methylene blue and 3-[hexadecyl(dimethyl)azaniumyl]propane-1-sulfonate.

74. The device, kit, system, or method of claim 71, wherein each droplet contains a methylene blue concentration of 0.01%-0.05% and a 3-[hexadecyl(dimethyl) azaniumyl]propane-1-sulfonate concentration of 0.5 mg/mL-2.0 mg/mL.

75. The device, kit, system, or method of any prior claim, further comprising a lysing agent configured to lyse red blood cells in the blood sample.

76. The device, kit, system, or method of claim 73, wherein the lysing agent is coated on one or both of the plates.

77. The device, kit, system, or method of claim 73, wherein the lysing agent is selected from the group consisting of ammonium chloride, organic quaternary ammonium surfactants, cyanide salts, and detergent.

78. The device, kit, system, or method of claim 73, wherein the lysing agent is selected from at least one of ammonium chloride, organic quaternary ammonium surfactants, cyanide salts, detergent, or any combination thereof.

79. The device, kit, system, or method of claim 75, wherein the detergent comprises 3-[hexadecyl(dimethyl)azaniumyl]propane-1-sulfonate.

80. The device kit, system, or method of claim 77, wherein the 3-[hexadecyl(dimethyl)azaniumyl]propane-1-sulfonate has a concentration of 0.5 mg/mL-2.0 mg/mL.

81. The device, kit, system, or method of claim 78, wherein the 3-[hexadecyl(dimethyl)azaniumyl]propane-1-sulfonate has a concentration of 1 mg/m L.

82. The device, kit, system, or method of claim 55, wherein steps (a)-(e) are performed in less than 10 minutes.

83. The device, kit, system, or method of claim 55, wherein steps (a)-(e) are performed in less than 5 minutes.

84. The device, kit, system, or method of claim 55, wherein steps (a)-(e) are performed in less than 1 minute.

85. The device, kit, system, or method of any prior claim, wherein the images are analyzed by machine learning.
